SEQUENCE LISTING (3 sequences)

SEQ ID NO: 1 - DNA, 1297 bp, Xenopus laevis
gacagtcggagccggcgctgcagcatcaaagggacttatcttggaggacttgtgaattctcatcctgccattgtggttactgagtctggttggacagaggaatgggcagcaacatgttcccggtgcctcttattgtcttttggggttttatcttggatggggcacttggctttgtcatgatgaccaactccaactccatcaagaatgtgccggcggcaccagcaggtcagcccattggctactaccctgtgagcgtcagtccggactccctatatgatattgccaacaagtaccaacctctggatgcctacccgctctacagttgcacggaagatgatgactgtgcccttgatgaattctgtcacagttccagaaacggcaactctctggtttgcttggcatgccggaaacgcagaaagcgttgcctgagggacgccatgtgctgcacaggcaactactgtagcaacggaatttgtgtccctgtggagcaagatcaagagcgcttccaacaccagggatacctggaagaaaccattctggaaaactataataatgctgatcatgcaacaatggatactcattccaaattaaccacgtccccatctggaatgcagccctttaaaggccgtgatggtgatgtttgcctccgatcaactgactgtgcgccaggtctatgctgtgcccgtcatttctggtcaaagatctgcaagccggtccttgatgaaggccaagtgtgcaccaagcacaggaggaaaggctctcacgggctagagattttccagcgttgtcactgcggtgccggactctcgtgccggttacagaaaggagaatttacaactgtccctaaaacatcgagacttcacacttgccaaagacactaagcgaggcctacagagcctgaaggaccttctctaaattaagctaattaagactttggtacctgcatgttattttctcagtttacatgaagtgctctggtcttccctgaacccggaagctgcgcaacttgtttctttttttgaggaacttcctaattaatgctaattacagtaaattactgtgttgtaaatactacgcaaggagacctgtaaaaactgtaaatacccgtgtatagaaagtgtacatgatcttctctattgtaacctgccaccttgtacattccgacgcgctcttccctttttatatatatatatatataaatatatattatattatgtagagtttacgtctagtatgtctgtatttttaattgaaataaaacatttctaaacttaaaaacaaaaaaaaaaaaaaaaaa

SEQ ID NO: 2 - DNA, 1226 bp, Mus Muscularis
gacccacgcgtccgtgcctgtttgcgtccttcggagatgatggttgtgtgtgcaccggcagctgtccggttcttggccgtgtttacaatgatggctctctgcagcctccctctgctaggagccagtgccaccttgaactcagttctcatcaattccaacgcgatcaagaacctgcccccaccgctgggtggtgctggggggcagccgggctctgctgtcagtgtggcgccgggagttctctatgagggcgggaacaagtaccagactcttgacaactaccagccctacccttgcgctgaagatgaggagtgcggctctgacgagtactgctccagccccagccgcggggcagccggcgtcggaggtgtacagatctgtctggcttgccgaaagcgcaggaagcgctgcatgacgcacgctatgtgctgccccgggaactactgcaaaaatggaatatgcatgccctctgaccacagccattttcctcgaggggaaattgaggaaagcatcattgaaaaccttggtaatgaccacaacgccgccgcgggggatggatatcccagaagaaccacactgacttcaaaaatatatcacaccaaaggacaagaaggctccgtctgcctccgatcatcagactgtgccgcagggctgtgttgtgcaagacacttctggtccaagatctgtaaacctgtccttaaagaaggtcaggtgtgcaccaagcacaaacggaaaggctcccacgggctggagatattccagcgctgttactgcggggaaggcctggcttgcaggatacagaaagatcaccatcaagccagcaattcttctaggctccacacctgccagagacactaaaccgacagtctaaatatgatggactctttttatctaatatatgctacgaaaatcctttatgatttgtcagctcaatcccaaggatgtaggaatcttcagtgtgtaattaagcattccgacaatactttccaaaagctctggagtgtaaggactttgtttcttgatggaactcccctgtgattgcagtaaattactgtgttgtaaatcctcagtgtggcacttacctgtaaatgcagcaaaacttttaattatttttctagaggtgtggtacattgccttgtttctcttgcatgtaaattttttttgtacacggttgattgtcttgactcataaatattctatattggagtagaaaaaaaaaaaaaaaaaaa

SEQ ID NO: 3 - DNA, 828 bp, Homo Sapiens
tggccccgcacgccaaaaattcggcacgagggtctggcactcagaggatgctctgaccttgaaagggtcctatctggagacgagggagtacaacgtgctgaatgtgtgcggttcagggagcatttggtaaccctgcatttgggagcagtgggcactaaccggttttggagaggtggacacataaggactgtgatcagcgcccgggtccaagagggcgggtacctggacctctgggtgcctcaccctctccccgaacccttcccacagccgtacccgtgcgcagaggacgaggagtgcggcactgatgagtactgcgctagtcccaccccgcggaggggaccgccggccgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaa

(19) United States
(12) Patent Application Publication
(10) Pub. No.: US 2012/0165270 A1
(43) Pub. Date: Jun. 28, 2012

(54) INHIBITION OF HAIR FOLLICLE GROWTH BY THE WNT INHIBITOR DKK1

(76) Inventors: Yeon Sook Choi, Philadelphia, PA (US); Sarah E. Millar, Bryn Mawr, PA (US); Thomas Andl, Nashville, TN (US)

(21) Appl. No.: 13/266,438

(22) PCT Filed: Apr. 27, 2010
(86) PCT No.: PCT/US10/32586

§ 371 Date: Mar. 14, 2012

Related U.S. Application Data
(60) Provisional application No. 61/173,123, filed on Apr. 27, 2009.

(51) Int. Cl. A61K 38/00; A61P 17/14; A61K 31/7088

(52) U.S. Cl. 514/20.7; 514/44 R

(57) ABSTRACT

The invention provides methods of inhibiting hair growth in a post-natal subject by contacting a matured hair follicle cell with a DKK polypeptide. The inhibitory mechanism induced by a DKK polypeptide results in a reversible, transient inhibition of hair growth.

### CROSS REFERENCE TO RELATED APPLICATIONS

This application claims priority to U.S. Provisional Patent Application 61/173,123, filed Apr. 27, 2009, which is incorporated by reference herein in its entirety.

### GOVERNMENT INTEREST

The work described herein was supported, in part, by a grant from the National Institute of Health, grant number R01-AR47709. The United States government may have certain rights in the invention.

### FIELD OF INVENTION

The invention relates to methods for inhibiting hair growth by contacting a hair follicle cell with a Wnt inhibitor, DKK1 in post-natal subjects wherein the inhibitory mechanism induced by a DKK polypeptide results in a reversible, transient inhibition of hair growth.

### BACKGROUND OF THE INVENTION

Hair follicles undergo cycles of growth (anagen), regression (catagen) and rest (telogen) throughout life. Cyclical hair growth is dependent on epithelial stem cells that reside in the permanent, bulge, region in the hair follicle outer root sheath. Signals from the dermal component of the hair follicle, known as the dermal papilla, are thought to initiate periods of hair growth by transiently stimulating division of the bulge stem cells. Each bulge stem cell is believed to give rise to one stem cell and one transiently amplifying daughter cell that migrates away from the bulge, towards the dermal papilla at the follicle base.

Further division of transiently amplifying cells gives rise to a population of relatively undifferentiated, rapidly dividing matrix cells that surround the dermal papilla. Progeny of the matrix give rise to cylindrical layers of cells that differentiate to form the hair shaft and its surrounding inner root sheath, that molds the hair shaft as it emerges from the follicle.

Hair removal describes any method of removing hair, especially from the human body. Depilation affects the part of the hair above the surface of the skin. The most common form of depilation is shaving. Another popular option is the use of chemical depilatories, which work by breaking the disulfide bonds that link the protein chains that give hair its strength, making the hair disintegrate. Epilation removal of the entire hair, including the part below the skin and is therefore longer-lasting. Some individuals may use waxing, sugaring, epilation devices, lasers, threading, intense pulsed light or electrology. Hair is also sometimes removed by plucking with tweezers.

Male-to-female transsexual women who are preparing for sex reassignment surgery usually remove their facial beard hair, typically either by electrolysis or laser, or a combination of the two procedures. While this is commonly done entirely before surgery, some patients will start the procedure before surgery, and finish a few months to several years afterwards, often due to cost. In addition, it is recommended by some surgeons that part of the pubic hair be removed prior to surgery as well, usually by electrolysis. Since the neovagina is created using the skin of the penis and part of the scrotum, which usually has active follicles, the hair is removed from these areas prior to surgery, in order for the genitals to be fashioned without the concern of hair growth inside of the neovagina. In some cases, the surgeon scrapes the underside of the skin to remove the follicles at or near the beginning of the surgery, eliminating any need for pre-surgical hair removal. Thus, negatively regulating hair-growth, namely preventing hair from growing, would be of tremendous use in medical procedures such as the above and for cosmetic purposes, and the present invention provides a manner in which this can be achieved.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee. The invention will be better understood from a reading of the detailed description taken in conjunction with the drawings in which like reference designators are used to designate like elements.

FIG. 1 shows rapid regression and reduced proliferation of hair follicles in mice induced to express ectopic Dkk1. Mice were placed on doxycycline chow at postnatal day 4. Skin sections of K5-rtTA; tetO-Dkk1 double transgenic (middle panels) and K14-Kremen1; K5-rtTA; tetO-Dkk1 triple transgenic (right panels) mice are compared with controls (left panels). (A-H) For histological analysis, triple, double transgenic and control mice were sacrificed at postnatal days 8 (A-C), 14 (D-F), or 20 (G and H). Sections were stained with hematoxylin and eosin. (1-L) Ki67 immunofluorescence was performed to determine cell proliferation using sections harvested at postnatal days 8 (I and J) and 14 (K and L). (M-N) Sections at postnatal day 8 were used for detection of BrdU incorporation. (O) The number of BrdU positive cells is shown as a percentage of the total number of DAPI-labeled nuclei. A total of 15-20 hair bulbs were counted for each time point. Each labeling method was repeated using samples from three independent mice, and the mean value was used for statistical analysis. Results are presented as mean±SEM. (P-Q). Expression of the cell cycle regulator cyclin D1 was determined by immunohistochemistry using sections harvested at postnatal day 8. Black arrows indicate the presence of cyclin D1 in hair matrix cells. A representative section is shown as an example in each case; similar results were obtained using samples from at least three independent mice. Scale bars: (A-H) 200 μm, (1-L) 100 μm, and (M, N, P, and Q) 50 μm.

FIG. 2 shows stem cell maintenance and persistence of hair follicle structures in inducible Dkk1 transgenic mice even during long-term Dkk1 expression. (A-L) Expression of Dkk1 was induced by doxycycline treatment at postnatal day 4, and skin samples were harvested at postnatal days 8 (A and B), 14 (C and D), 20 (E and F), and 183 (G-L), respectively. Skin sections of K5-rtTA; tetO-Dkk1 double transgenic (right panels) mice are compared with controls (left panels). (A-H) Immunofluorescence for the keratin 15 stem cell marker shows the presence of keratin 15 in K5-rtTA; tetO-Dkk1 double transgenic mice even during long periods of Dkk1 expression. The signal appears as green fluorescence and is indicated by yellow arrows in each panel. Insets in panels A-F represent higher magnification photographs of the regions indicated by arrows in each panel. (I-J) Skin sections at postnatal day 183 were stained with hematoxylin and eosin for histological analysis. Black arrows indicate the presence of hair follicles in control and double transgenic skin. (K-L) Immunofluorescence for the p63 basal cell marker shows no significant difference in p63 expression between control and induced Dkk1 transgenic mice. (M-U) Dkk1 expression was induced at postnatal day 7, and dorsal skin was harvested at postnatal day 102. Skin sections of K5-rtTA; tetO-Dkk1 double transgenic (middle panels) and K14-Kremen1; K5-rtTA; tetO-Dkk1 triple transgenic (right panels) mice are compared with controls (left panels). Histological analysis (P-R) and detection of keratin 15 (S-U) are shown. Scale bars: (A, B, C, I, and J) 200 μm, (D-H and P-R) 100 μm, and (K-L and S-U) 50 μm.

FIG. 3 shows that plucking-induced anagen is blocked in induced K5-rtTA; tetO-Dkk1 double transgenic mice. Doxycycline induction of Dkk1 expression was initiated in telogen, either at postnatal day 18 (A-K; P18-57) or at postnatal day 51 (L-W; P51-57), and hair plucking was performed at postnatal day 52 (A-K) or at postnatal day 54 (L-W) to induce a new anagen growth phase. All mice were sacrificed at postnatal day 57, 5 days (A-K) or 3 days (L-W) after depilation. (A-B) Histological staining with hematoxylin and eosin reveals that hair follicles in K5-rtTA; tetO-Dkk1 double transgenic mice are arrested in early anagen. (C-D) Expression of (β-catenin detected by immunohistochemistry. Nuclear localized β-catenin is observed in the matrix cells (black arrows) as well as the dermal papilla (a dashed line circle) of control hair follicles (C). K5-rtTA; tetO-Dkk1 double transgenic hair follicles show no nuclear β-catenin in the matrix cells (D). The blue arrow indicates β-catenin expression at the cell membrane. (E-F) A TUNEL assay was performed to detect apoptotic cells. No apoptotic cells were seen in either control or double transgenic skin sections. (G-K) Cell proliferation was determined by immunofluorescence for Ki67 (G-H, green) and BrdU (1-J, red). BrdU incorporation was quantified as a percentage of the total number of DAPI-labeled nuclei (K). Data are presented as mean±SEM. (L-M) K5-rtTA; tetO-Dkk1 double transgenic and control mice were crossed with Conductin-lacZ, Wnt reporter mice (Lustig et al., 2002, Mol Cell Biol 22, 1184-1193). Skin sections of induced control mice carrying Conductin-lacZ (L) and K5-rtTA; tetO-Dkk1 transgenic mice carrying Conductin-lacZ (M) were stained for β-galactosidase activity to visualize Wnt signaling activity. A blue signal indicating β-galactosidase activity is seen in the hair bulb of control but not K5-rtTA; tetO-Dkk1 hair follicles (L, black arrow). (N-O) Immunohistochemistry for cyclin D1 shows diminished expression of cyclin D1 in K5-rtTA; tetO-Dkk1 double transgenic hair follicles compared with controls. The signal appears in brown color and is indicated by black arrows. (P-W) Skin sections stained with pSmad1/5/8 (P and Q, red), BMP6 (R and S, red), GATA3 (T and U, green), and AE13 (V and W, green) antibodies show absence of pSmad1/5/8, BMP6, GATA3, and AE13 proteins in K5-rtTA; tetO-Dkk1 double transgenic hair follicles during plucking-induced early anagen. Yellow arrows indicate positive signals in the dermal papillae of control hair follicles (P and R). Scale bars: (A-B and E-F) 100 μm and (C-D, G-J, and L-W) 50 μm.

FIG. 4 shows that the effects Dkk1 on hair growth in K5-rtTA; tetO-Dkk1 double transgenic mice are reversible. (A-H) Control and K5-rtTA; tetO-Dkk1 double transgenic mice were maintained on doxycycline for 10 months (Postnatal day 15-294), removed from doxycycline for 1 month (P295-326), and then placed back on doxycycline for another 3.5 months (P327-435). Doxycycline chow was withdrawn at postnatal day 435 and hair plucking was performed at postnatal day 438. Skin samples were harvested 21 days after depilation, and were used for histological analysis (A and B), keratin 15 (C and D) and Ki67 (E and F) detection, and TUNEL assay (G and H). Insets in panels C-H represent higher magnification photographs of the regions indicated by arrows in each panel. (1-N) Control and K5-rtTA; tetO-Dkk1 double transgenic mice were placed on doxycycline chow at postnatal day 21, and skin biopsies were performed at postnatal day 475 (approximately 15.5 months). In contrast with control mice (I), K5-rtTA; tetO-Dkk1 double transgenic mice are completely hairless (J). Histological examination (K and L) shows maintenance of hair follicle structures in double transgenic skin (L). Immunofluorescence for keratin 15 (M and N) reveals persistence of hair follicle stem cells in double transgenic skin (N). (O-Z) K5-rtTA; tetO-Dkk1 transgenic mice maintained on doxycycline from postnatal day 21 were removed from doxycycline chow at postnatal day 475. Skin biopsies were performed at postnatal day 475 (on doxycycline; 0, Q, S, U, W, and Y) and at postnatal day 489 (off doxycycline; P, R, T, V, X, and Z), 14 days after doxycycline withdrawal. Hair regrowth in double transgenic mice was observed following removal of doxycycline (P). In situ hybridization for Dkk1 reveals ectopic Dkk1 expression in the epidermis and hair follicles (black arrow) of double transgenic skin on doxycycline chow (Q) and the absence of Dkk1 following doxycycline withdrawal (R). Skin sections were stained with β-catenin (S and T, brown), cyclin D1 (U and V, brown), Ki67 (W and X, green), and AE13 (Y and Z, green) antibodies. In contrast with double transgenic hair follicles from mice on doxycycline, K5-rtTA; tetO-Dkk1 transgenic hair follicles after removal of doxycycline show expression of nuclear β-catenin (T, black arrow), cyclin D1 (V, black arrow), Ki67 (X), and AE13 (Z) proteins. Yellow arrows indicate positive signals of green-fluorescence in each panel. Scale bars: (A-H) 200 μm, (K-M and O—R) 100 μm, and (N and S-Z) 50 μm.

FIG. 5 shows that ectopic expression of Kremen1 alone has no effect on hair follicle growth. (A) RT-PCR analysis of reverse-transcribed total RNA from dorsal skin was performed using primers specific for mouse Kremen1. As a control, GAPDH was amplified. PCR was performed with RNA samples processed with (+) or without (−) reverse transcriptase. (B-C) Histological analysis of skin sections from K5-rtTA; K14-Kremen1 transgenic (C) and control (B) mice at postnatal day 12. Scale Bar: (B-C) 200 μm.

FIG. 6 shows that the plucking-induced anagen phase is effectively blocked in hair follicles of K14-Kremen1; K5-rtTA; tetO-Dkk1 triple transgenic mice. Mice were placed on doxycycline chow at postnatal day 51, and hair plucking was performed at postnatal day 54. Dorsal skin was harvested at postnatal day 57, 3 days after depilation. Skin sections of K5-rtTA; tetO-Dkk1 double transgenic (middle panels) and K14-Kremen1; K5-rtTA; tetO-Dkk1 triple transgenic (right panels) mice are compared with controls (left panels). Histological analysis (A-C) and immunofluorescence for the keratin 15 stem cell marker (D-F) are shown. (G-I) TUNEL assay was performed to measure apoptosis. Proliferation was examined both by staining for the proliferation antigen Ki67 (J-L) and by quantifying BrdU incorporation (M). BrdU incorporation was quantified as a percentage of the total number of DAPI-labeled nuclei. Data are presented as mean±SEM. Scale Bars: (A-C) 100 μm and (D-L) 50 μm.

### SUMMARY OF THE INVENTION

In one embodiment, the present invention provides a method for transiently or reversibly inhibiting hair growth in a post-natal subject without damaging skin or damaging a hair follicle, comprising the step of contacting a LRP-family, Wnt-associated receptor in a matured hair follicle cell with a DKK polypeptide or its peptidomimetic analog, thereby inhibiting hair growth in a post-natal subject.

In another embodiment, the present invention provides a method for modulating hair growth in a post-natal subject, comprising the step of contacting a LRP-family, Wnt-associated receptor in a matured hair follicle cell with a DKK polypeptide or its peptidomimetic analog, thereby inhibiting hair growth in a post-natal subject.

In one embodiment, the present invention provides a method for transiently or reversibly inhibiting hair growth in a post-natal subject without damaging skin or damaging a hair follicle, comprising the step of contacting a matured hair follicle cell with a DNA construct comprising the nucleic acid sequence encoding a DKK polypeptide, thereby inhibiting hair growth in a post-natal subject.

In another embodiment, the present invention provides a method for modulating hair growth in a post-natal subject, comprising the step of contacting a matured hair follicle cell with a DNA construct comprising the nucleic acid sequence encoding a DKK polypeptide, thereby inhibiting hair growth in a post-natal subject.

In another embodiment, the present invention provides a method for modulating hair growth in a post-natal subject without damaging skin or damaging a hair follicle, comprising the step of inhibiting Wnt/beta-catenin signaling in a matured hair follicle cell of the subject with a polypeptide capable of binding to LRP-family, Wnt-associated receptor, thereby modulating hair growth in a post-natal subject.

In one embodiment, the invention provides a method of temporarily removing hair on a subject without damaging skin or damaging a hair follicle, comprising the step of contacting skin of the subject with a topical composition comprising a DKK polypeptide or its peptidomimetic, thereby blocking LRP-family, Wnt-associated receptor in a mature hair follicle cell in the skin, thus temporarily removing hair on a subject.

In another embodiment, the invention provides a method of temporarily inhibiting hair growth on a subject without damaging skin or damaging a hair follicle, comprising the step of contacting skin of the subject with a topical composition comprising DKK polypeptide or its peptidomimetic, thereby blocking LRP-family, Wnt-associated receptor in a mature hair follicle cell in the skin, thus temporarily removing hair on a subject.

In one embodiment, the invention provides a method of inhibiting hair growth in a mature hair follicle, comprising the step of inducing the expression of a secreted Wnt inhibitor in a hair follicle cell, thereby inhibiting hair growth in a hair follicle.

In another embodiment, the invention provides a method of inhibiting hair growth of a hair follicle in a post-natal subject, comprising the step of inducing the expression of a secreted Wnt inhibitor in a hair follicle cell, thereby preventing hair growth of a hair follicle in a subject.

In one embodiment, the invention provides a method of inhibiting hair growth of a hair follicle in a post-natal subject, comprising the step of contacting the hair follicle during anagen phase of the hair follicle cell cycle with a DKK polypeptide or its peptidomimetic analog, thereby inhibiting hair growth of a hair follicle.

In another embodiment, the invention provides a method of inhibiting the proliferation of a hair follicle cell in a post-natal subject without increasing cell death of the hair follicle cells, comprising the step of contacting the hair follicle cell with a DKK polypeptide or its peptidomimetic analog, thereby inhibiting the proliferation of a hair follicle cell in a post-natal subject.

In one embodiment, the invention provides a method of inhibiting hair growth on a hair follicle in a post-natal subject without alteration in stem cell maintenance in the hair follicle, comprising the step of contacting the hair follicle during anagen phase of the hair follicle cell cycle with a DKK polypeptide or its peptidomimetic analog, thereby inhibiting hair growth on a hair follicle.

In another embodiment, the invention provides a method of maintaining a bald spot on a skin in a post-natal subject without damaging skin or damaging a hair follicle, comprising the steps of: contacting a LRP-family, Wnt-associated receptor in a matured hair follicle cell within the bald spot with a DKK polypeptide or its peptidomimetic analog; and removing hair within the spot, thereby maintaining a bald spot on a skin in a post-natal subject.

In one embodiment, the invention provides a method of inhibiting hair cortex formation in a post-natal subject without damaging skin or damaging a hair follicle, comprising the step of contacting a LRP-family, Wnt-associated receptor in a matured hair follicle cell with a DKK polypeptide or its peptidomimetic analog, thereby inhibiting hair cortex formation in a post-natal subject.

In another embodiment, the invention provides a method of reversibly inhibiting hair growth in a post-natal subject without damaging skin or damaging the hair follicle, comprising the step of contacting a LRP-family, Wnt-associated receptor in a mature hair follicle cell with a DKK polypeptide or its peptidomimetic analog, thereby reversibly inhibiting hair growth in a post-natal subject.

In one embodiment, the invention provides a method of treating, inhibiting or suppressing or ameliorating symptoms associated with hirsutism or hypertrichosis in a subject, comprising the step of administering to the subject a composition comprising a DKK polypeptide or its peptidomimetic analog, thereby binding a LRP-family, Wnt-associated receptor and reversibly inhibiting hair growth in the subject.

In another embodiment, the invention provides a method of treating, inhibiting or suppressing or ameliorating symptoms associated with hirsutism or hypertrichosis in a subject, comprising the step of administering to the subject a composition comprising a DNA construct encoding a secreted Wnt inhibitor, thereby binding a LRP-family, Wnt-associated receptor and reversibly inhibiting hair growth in the subject.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION OF THE INVENTION

In one embodiment, a Dickkopf homolog 1 (Xenopus laevis), also known as DKK1, is a human gene and this gene encodes a protein that is a member of the dickkopf family, wherein in other embodiments, it is a secreted protein with two cysteine rich regions and is involved in embryonic development through its inhibition of the WNT signaling pathway.

In one embodiment, provided herein a method of inhibiting hair growth in a post-natal subject, comprising the step of contacting a matured hair follicle cell with a DKK polypeptide, thereby inhibiting hair growth in a post-natal subject. In another embodiment, provided herein a method of inhibiting hair growth in a post-natal subject, comprising the step of contacting a matured hair follicle cell with a DNA molecule comprising the nucleic acid sequence encoding a DKK polypeptide, thereby inhibiting hair growth in a post-natal subject.

In another embodiment, provided herein a method of transiently inhibiting hair growth in a post-natal subject, comprising the step of inhibiting Wnt/beta-catenin signaling in a matured hair follicle cell of a post-natal subject, thereby transiently inhibiting hair growth in a post-natal subject. In another embodiment, provided herein a method of reversibly inhibiting hair growth in a post-natal subject, comprising the step of inhibiting Wnt/beta-catenin signaling in a matured hair follicle cell of a post-natal subject, thereby reversibly inhibiting hair growth in a post-natal subject.

In another embodiment, inhibiting Wnt/beta-catenin signaling is transiently inhibiting Wnt/beta-catenin signaling. In another embodiment, inhibiting Wnt/beta-catenin signaling is reversibly inhibiting Wnt/beta-catenin signaling. In another embodiment, inhibiting Wnt/beta-catenin signaling comprises blocking a LRP-family, Wnt-associated receptor. In another embodiment, inhibiting Wnt/beta-catenin signaling comprises contacting a matured hair follicle cell with a DKK polypeptide.

In another embodiment, provided herein a method of blocking hair growth in a post-natal subject, comprising the step of contacting a matured hair follicle cell with a DKK polypeptide, thereby inhibiting hair growth in a post-natal subject. In another embodiment, provided herein a method of delaying hair growth in a post-natal subject, comprising the step of contacting a matured hair follicle cell with a DKK polypeptide, thereby inhibiting hair growth in a post-natal subject. In another embodiment, provided herein a method of slowing hair growth in a post-natal subject, comprising the step of contacting a matured hair follicle cell with a DKK polypeptide, thereby inhibiting hair growth in a post-natal subject.

In another embodiment, provided herein a method of transiently inhibiting hair growth in a post-natal subject, comprising the step of contacting a matured hair follicle cell with a DKK polypeptide, thereby transiently inhibiting hair growth in a post-natal subject. In another embodiment, provided herein a method of transiently inhibiting hair growth is a method of temporarily inhibiting hair growth. In another embodiment, provided herein a method of inhibiting hair growth for a limited period of time in a post-natal subject, comprising the step of contacting a matured hair follicle cell with a DKK polypeptide.

In another embodiment, provided herein a method of inhibiting hair growth for a period of 1-365 days in a post-natal subject, comprising the step of contacting a matured hair follicle cell with a DKK polypeptide. In another embodiment, provided herein a method of inhibiting hair growth for a period of 1-10 days in a post-natal subject, comprising the step of contacting a matured hair follicle cell with a DKK polypeptide. In another embodiment, provided herein a method of inhibiting hair growth for a period of 7-14 days in a post-natal subject, comprising the step of contacting a matured hair follicle cell with a DKK polypeptide. In another embodiment, provided herein a method of inhibiting hair growth for a period of 10-30 days in a post-natal subject, comprising the step of contacting a matured hair follicle cell with a DKK polypeptide. In another embodiment, provided herein a method of inhibiting hair growth for a period of 30-60 days in a post-natal subject, comprising the step of contacting a matured hair follicle cell with a DKK polypeptide. In another embodiment, provided herein a method of inhibiting hair growth for a period of 50-90 days in a post-natal subject, comprising the step of contacting a matured hair follicle cell with a DKK polypeptide. In another embodiment, provided herein a method of inhibiting hair growth for a period of 80-120 days in a post-natal subject, comprising the step of contacting a matured hair follicle cell with a DKK polypeptide. In another embodiment, provided herein a method of inhibiting hair growth for a period of 100-150 days in a post-natal subject, comprising the step of contacting a matured hair follicle cell with a DKK polypeptide. In another embodiment, provided herein a method of inhibiting hair growth for a period of 125-175 days in a post-natal subject, comprising the step of contacting a matured hair follicle cell with a DKK polypeptide. In another embodiment, provided herein a method of inhibiting hair growth for a period of 150-250 days in a post-natal subject, comprising the step of contacting a matured hair follicle cell with a DKK polypeptide. In another embodiment, provided herein a method of inhibiting hair growth for a period of 200-300 days in a post-natal subject, comprising the step of contacting a matured hair follicle cell with a DKK polypeptide. In another embodiment, provided herein a method of inhibiting hair growth for a period of 270-365 days in a post-natal subject, comprising the step of contacting a matured hair follicle cell with a DKK polypeptide.

In another embodiment, provided herein a method of inhibiting hair growth for a period of 1-50 years in a post-natal subject, comprising the step of contacting a cell or a tissue comprising matured hair follicle with a DKK polypeptide. In another embodiment, provided herein a method of inhibiting hair growth for a period of 1-2 years in a post-natal subject, comprising the step of contacting a matured hair follicle cell with a DKK polypeptide. In another embodiment, provided herein a method of inhibiting hair growth for a period of 1-5 years in a post-natal subject, comprising the step of contacting a matured hair follicle cell with a DKK polypeptide. In another embodiment, provided herein a method of inhibiting hair growth for a period of 5-10 years in a post-natal subject, comprising the step of contacting a matured hair follicle cell with a DKK polypeptide. In another embodiment, provided herein a method of inhibiting hair growth for a period of 7-15 years in a post-natal subject, comprising the step of contacting a matured hair follicle cell with a DKK polypeptide. In another embodiment, provided herein a method of inhibiting hair growth for a period of 10-20 years in a post-natal subject, comprising the step of contacting a matured hair follicle cell with a DKK polypeptide. In another embodiment, provided herein a method of inhibiting hair growth for a period of 20-50 years in a post-natal subject, comprising the step of contacting a matured hair follicle cell with a DKK polypeptide.

In another embodiment, provided herein a method of reversibly inhibiting hair growth in a post-natal subject, comprising the step of contacting a matured hair follicle cell with a DKK polypeptide, thereby transiently inhibiting hair growth in a post-natal subject. In another embodiment, the effect of Dkk1 polypeptide is limited to Dkk1 stability. In another embodiment, the effect of Dkk1 polypeptide is limited to Dkk1 half-life. In another embodiment, the effect of Dkk1 protein in a hair follicle cell is transient. In another embodiment, the effect of Dkk1 on Wnt pathway is transient. In another embodiment, the inhibitory effect of Dkk1 on Wnt pathway in a hair follicle cell is transient.

In another embodiment, the term matured hair follicle includes a hair follicle of a post-natal animal. In another embodiment, the term matured hair follicle includes a hair follicle of a post-natal subject. In another embodiment, the term matured hair follicle includes a hair follicle of a post-natal human subject. In another embodiment, the term matured hair follicle includes a differentiated hair follicle cell. In another embodiment, the term matured hair follicle includes a non-embryonic hair follicle cell. In another embodiment, the term matured hair follicle includes a hair follicle cell comprising a hair shaft. In another embodiment, the term matured hair follicle includes a hair follicle cell comprising a dermal papilla.

In another embodiment, a DKK polypeptide is a mammalian DKK polypeptide. In another embodiment, a DKK polypeptide is a human DKK polypeptide. In another embodiment, a DKK polypeptide is a Dkk1 polypeptide. In another embodiment, a DKK polypeptide is a mammalian Dkk1 polypeptide. In another embodiment, a DKK polypeptide is a human Dkk1 polypeptide. In another embodiment, a DKK polypeptide is an inhibitor protein of the wnt signal pathway.

In another embodiment, a Dkk1 polypeptide is encoded by a DNA molecule comprising: gacagtcggagccggcgctgcagcatcaaagggacttatcttggaggacttgtgaattctcatcctgccattgtggttactgagtctggttggacaga ggaatgggcagcaacatgttcccggtgcctcttattgtcttttggggttttatcttggatggggcacttggctttgtcatgatgaccaactccaactccat caagaatgtgccggcggcaccagcaggtcagcccattggctactaccctgtgagcgtcagtccggactccctatatgatattgccaacaagtacca acctctggatgcctacccgctctacagttgcacggaagatgatgactgtgcccttgatgaattctgtcacagttccagaaacggcaactctctggtttg cttggcatgccggaaacgcagaaagcgttgcctgagggacgccatgtgctgcacaggcaactactgtagcaacggaatttgtgtccctgtggagc aagatcaagagcgcttccaacaccagggatacctggaagaaaccattctggaaaactataataatgctgatcatgcaacaatggatactcattccaa attaaccacgtccccatctggaatgcagccctttaaaggccgtgatggtgatgtttgcctccgatcaactgactgtgcgccaggtctatgctgtgccc gtcatttctggtcaaagatctgcaagccggtccttgatgaaggccaagtgtgcaccaagcacaggaggaaaggctctcacgggctagagattttcc agcgttgtcactgcggtgccggactctcgtgccggttacagaaaggagaatttacaactgtccctaaaacatcgagacttcacacttgccaaagaca ctaagcgaggcctacagagcctgaaggaccttctctaaattaagctaattaagactttggtacctgcatgttattttctcagtttacatgaagtgctctgg tcttccctgaacccggaagctgcgcaacttgtttctttttttgaggaacttcctaattaatgctaattacagtaaattactgtgttgtaaatactacgcaagg agacctgtaaaaactgtaaatacccgtgtatagaaagtgtacatgatcttctctattgtaacctgccaccttgtacattccgacgcgctcttccctttttat atatatatatatataaatatatattatattatgtagagtttacgtctagtatgtctgtatttttaattgaaataaaacatttctaaacttaaaaacaaaaaaaaaa aaaaaaaa (SEQ ID NO: 1). In another embodiment, SEQ ID NO: 1 is a DNA molecule encoding Xenopus laevis Dkk1. In another embodiment, Dkk1 is encoded by a DNA molecule related to SEQ ID NO: 1 due to the degeneracy of the genetic code.

In another embodiment, a Dkk1 polypeptide is encoded by a DNA molecule comprising: gacccacgcgtccgtgcctgtttgcgtccttcggagatgatggttgtgtgtgcaccggcagctgtccggttcttggccgtgtttacaatgatggctctc tgcagcctccctctgctaggagccagtgccaccttgaactcagttctcatcaattccaacgcgatcaagaacctgcccccaccgctgggtggtgctg gggggcagccgggctctgctgtcagtgtggcgccgggagttctctatgagggcgggaacaagtaccagactcttgacaactaccagccctaccct tgcgctgaagatgaggagtgcggctctgacgagtactgctccagccccagccgcggggcagccggcgtcggaggtgtacagatctgtctggctt gccgaaagcgcaggaagcgctgcatgacgcacgctatgtgctgccccgggaactactgcaaaaatggaatatgcatgccctctgaccacagcca ttttcctcgaggggaaattgaggaaagcatcattgaaaaccttggtaatgaccacaacgccgccgcgggggatggatatcccagaagaaccacac tgacttcaaaaatatatcacaccaaaggacaagaaggctccgtctgcctccgatcatcagactgtgccgcagggctgtgttgtgcaagacacttctg gtccaagatctgtaaacctgtccttaaagaaggtcaggtgtgcaccaagcacaaacggaaaggctcccacgggctggagatattccagcgctgtta ctgcggggaaggcctggcttgcaggatacagaaagatcaccatcaagccagcaattcttctaggctccacacctgccagagacactaaaccgaca gtctaaatatgatggactctttttatctaatatatgctacgaaaatcctttatgatttgtcagctcaatcccaaggatgtaggaatcttcagtgtgtaattaa gcattccgacaatactttccaaaagctctggagtgtaaggactttgtttcttgatggaactcccctgtgattgcagtaaattactgtgttgtaaatcctcag tgtggcacttacctgtaaatgcagcaaaacttttaattatttttctagaggtgtggtacattgccttgtttctcttgcatgtaaattttttttgtacacggttgat tgtcttgactcataaatattctatattggagtagaaaaaaaaaaaaaaaaaaa (SEQ ID NO: 2). In another embodiment, SEQ ID NO: 2 is a DNA molecule encoding Mus Muscularis Dkk1. In another embodiment, Dkk1 is encoded by a DNA molecule related to SEQ ID NO: 2 due to the degeneracy of the genetic code.

In another embodiment, a Dkk1 polypeptide is encoded by a DNA molecule comprising: tggccccgcacgccaaaaattcggcacgagggtctggcactcagaggatgctctgaccttgaaagggtcctatctggagacgagggagtacaac gtgctgaatgtgtgcggttcagggagcatttggtaaccctgcatttgggagcagtgggcactaaccggttttggagaggtggacacataaggactgt gatcagcgcccgggtccaagagggcgggtacctggacctctgggtgcctcaccctctccccgaacccttcccacagccgtacccgtgcgcagag gacgaggagtgcggcactgatgagtactgcgctagtcccaccccgcggaggggaccgccggccgtgcaaatctgtctcgcctgcaggaagcgc cgaaaacgctgcatgcgtcacgctatgtgctgccccgggaattactgcaaaaatggaatatgtgtgtcttctgatcaaaatcatttccgaggagaaat tgaggaaaccatcactgaaagctttggtaatgatcatagcaccttggatgggtattccagaagaaccaccttgtcttcaaaaatgtatcacaccaaag gacaagaaggttctgtttgtctccggtcatcagactgtgcctcaggattgtgttgtgctagacacttctggtccaagatctgtaaacctgtcctgaaaga aggtcaagtgtgtaccaagcataggagaaaaggctctcatggactagaaatattccagcgttgttactgtggagaaggtctgtcttgccggatacag aaagatcaccatcaagccagtaattcttctaggcttcacacttgtcagagacactaa (SEQ ID NO: 3). In another embodiment, SEQ ID NO: 3 is a DNA molecule encoding Homo Sapiens Dkk1. In another embodiment, Dkk1 is encoded by a DNA molecule related to SEQ ID NO: 3 due to the degeneracy of the genetic code.

In another embodiment, the DNA molecule encoding a Dkk1 polypeptide is at least 70% homologous to the DNA sequence of SEQ ID NO: 1. In another embodiment, the DNA molecule encoding a Dkk1 polypeptide is at least 80% homologous to the DNA sequence of SEQ ID NO: 1. In another embodiment, the DNA molecule encoding a Dkk1 polypeptide is at least 85% homologous to the DNA sequence of SEQ ID NO: 1. In another embodiment, the DNA molecule encoding a Dkk1 polypeptide is at least 90% homologous to the DNA sequence of SEQ ID NO: 1. In another embodiment, the DNA molecule encoding a Dkk1 polypeptide is at least 95% homologous to the DNA sequence of SEQ ID NO: 1.

In one embodiment, provided herein is a method of inhibiting hair growth in a post-natal subject, comprising the step of contacting a LRP-family, Wnt-associated receptor in a matured hair follicle cell with a DKK polypeptide or its peptidomimetic analog, thereby inhibiting hair growth in a post-natal subject.

In one embodiment, provided herein is a method of inhibiting hair growth in a post-natal subject without damaging skin or damaging a hair follicle, comprising the step of contacting a LRP-family, Wnt-associated receptor in a matured hair follicle cell with a DKK polypeptide or its peptidomimetic analog, thereby inhibiting hair growth in a post-natal subject.

In another embodiment, provided herein is a method of modulating hair growth in a post-natal subject, comprising the step of contacting a LRP-family, Wnt-associated receptor in a matured hair follicle cell with a DKK polypeptide or its peptidomimetic analog, thereby inhibiting hair growth in a post-natal subject.

In one embodiment, provided herein is a method of inhibiting hair growth in a post-natal subject without damaging skin or damaging a hair follicle, comprising the step of contacting a matured hair follicle cell with a DNA construct comprising the nucleic acid sequence encoding a DKK polypeptide, thereby inhibiting hair growth in a post-natal subject.

In another embodiment, provided herein is a method of modulating hair growth in a post-natal subject, comprising the step of contacting a matured hair follicle cell with a DNA construct comprising the nucleic acid sequence encoding a DKK polypeptide, thereby inhibiting hair growth in a post-natal subject.

In another embodiment, provided herein is a method of modulating hair growth in a post-natal subject without damaging skin or damaging a hair follicle, comprising the step of inhibiting Wnt/beta-catenin signaling in a matured hair follicle cell of the subject with a polypeptide capable of binding to LRP-family, Wnt-associated receptor, thereby modulating hair growth in a post-natal subject.

In one embodiment, provided herein is a method of temporarily removing hair on a subject without damaging skin or damaging a hair follicle, comprising the step of contacting skin of the subject with a topical composition comprising a DKK polypeptide or its peptidomimetic, thereby blocking LRP-family, Wnt-associated receptor in a mature hair follicle cell in the skin, thus temporarily removing hair on a subject.

In another embodiment, provided herein is a method of temporarily inhibiting hair growth on a subject without damaging skin or damaging a hair follicle, comprising the step of contacting skin of the subject with a topical composition comprising DKK polypeptide or its peptidomimetic, thereby blocking LRP-family, Wnt-associated receptor in a mature hair follicle cell in the skin, thus temporarily removing hair on a subject.

In one embodiment, provided herein is a method of inhibiting hair growth in a mature hair follicle, comprising the step of inducing the expression of a secreted Wnt inhibitor in a hair follicle cell, thereby inhibiting hair growth in a hair follicle.

In another embodiment, provided herein is a method of inhibiting hair growth of a hair follicle in a post-natal subject, comprising the step of inducing the expression of a secreted Wnt inhibitor in a hair follicle cell, thereby preventing hair growth of a hair follicle in a subject.

In one embodiment, provided herein is a method of inhibiting hair growth of a hair follicle in a post-natal subject, comprising the step of contacting the hair follicle during anagen phase of the hair follicle cell cycle with a DKK polypeptide or its peptidomimetic analog, thereby inhibiting hair growth of a hair follicle. In one embodiment, Dkk1 expression during anagen of the embryonic hair cycle inhibits hair follicle growth, wherein in other embodiments, induction of Dkk1 during anagen of embryonic hair cycle causes inhibition of hair follicle cell proliferation as demonstrated in Example 1 herein.

In another embodiment, provided herein is a method of inhibiting the proliferation of a hair follicle cell in a post-natal subject without increasing cell death of the hair follicle cells, comprising the step of contacting the hair follicle cell with a DKK polypeptide or its peptidomimetic analog, thereby inhibiting the proliferation of a hair follicle cell in a post-natal subject. In another embodiment, rapid entry of Dkk1-expressing hair follicles into the catagen regression phase of the hair follicle growth cycle results in reduced cell proliferation as demonstrated in the Examples. In some embodiments, Dkk1 expression caused regression of hair follicles wherein in other embodiments this regression is reversible as shown in Example 4.

In one embodiment, provided herein is a method of inhibiting hair growth on a hair follicle in a post-natal subject without alteration in stem cell maintenance in the hair follicle, comprising the step of contacting the hair follicle during anagen phase of the hair follicle cell cycle with a DKK polypeptide or its peptidomimetic analog, thereby inhibiting hair growth on a hair follicle. In one embodiment, hair growth inhibition is reversible because stem cells are not affected when using the methods comprising contacting a hair follicle with a DKK polypeptide, as is demonstrated in Examples 2 and 4 herein. In other embodiments, hair follicle structures and the epithelial bulge stem cell compartment are maintained even during long term expression of a DKK polypeptide as demonstrated in Example 2.

In another embodiment, provided herein is a method of maintaining a bald spot on a skin in a post-natal subject without damaging skin or damaging a hair follicle, comprising the steps of: contacting a LRP-family, Wnt-associated receptor in a matured hair follicle cell within the bald spot with a DKK polypeptide or its peptidomimetic analog; and removing hair within the spot, thereby maintaining a bald spot on a skin in a post-natal subject.

In one embodiment, provided herein is a method of inhibiting hair cortex formation in a post-natal subject without damaging skin or damaging a hair follicle, comprising the step of contacting a LRP-family, Wnt-associated receptor in a matured hair follicle cell with a DKK polypeptide or its peptidomimetic analog, thereby inhibiting hair cortex formation in a post-natal subject.

In another embodiment, provided herein is a method of reversibly inhibiting hair growth in a post-natal subject without damaging skin or damaging the hair follicle, comprising the step of contacting a LRP-family, Wnt-associated receptor in a mature hair follicle cell with a DKK polypeptide or its peptidomimetic analog, thereby reversibly inhibiting hair growth in a post-natal subject.

In one embodiment, provided herein is a method of treating, inhibiting or suppressing or ameliorating symptoms associated with hirsutism or hypertrichosis in a subject, comprising the step of administering to the subject a composition comprising a DKK polypeptide or its peptidomimetic analog, thereby binding a LRP-family, Wnt-associated receptor and reversibly inhibiting hair growth in the subject.

In another embodiment, provided herein is a method of treating, inhibiting or suppressing or ameliorating symptoms associated with hirsutism or hypertrichosis in a subject, comprising the step of administering to the subject a composition comprising a DNA construct encoding a secreted Wnt inhibitor, thereby binding a LRP-family, Wnt-associated receptor and reversibly inhibiting hair growth in the subject.

In one embodiment, the LRP-family wnt-associated receptors are LRP co-receptors (LRP5/6, Arrow) referring to single transmembrane proteins that comprise a subfamily of LDL-receptor related proteins and play an essential role in the canonical β-catenin pathway. In another embodiment, the intracellular domains of LRP5 and Arrow bind to Axin, which is translocated and destabilized by Wnt. LRP has a large extracellular domain that contains four EGF repeats and three LDLR repeats.

In one embodiment, the term “peptidomimetic” refers to a compound containing non-peptidic structural elements that is capable of mimicking or modulating the biological action(s) of a natural parent peptide, such as DKK in one embodiment.

In another embodiment, provided herein is a method of inhibiting hair growth in a post-natal subject, comprising the step of contacting a matured hair follicle cell with a DNA construct comprising the nucleic acid sequence encoding a DKK polypeptide, thereby inhibiting hair growth in a post-natal subject. In one embodiment, the term DNA construct and DNA molecule is interchangeable.

In one embodiment, the term “DNA construct” or “DNA molecule” refers to an expression or transformation construct. The DNA construct comprises at least a shortened DNA sequence, which encodes a form of the DKK protein, preferably in combination with appropriate regulatory sequences, which include a promoter, a signal sequence with or without a carrier sequence, and a terminator sequence. The DNA construct includes at least the DNA sequences, which are essential for competent expression and secretion of the form of the desired DKK polypeptide. The DNA constructs can be provided as an expression cassette in one embodiment, or as an expression plasmid. in another embodiment.

In another embodiment, an expression plasmid of the DNA construct may further contain plasmid elements and reporter gene sequences for replication and selection in E. coli. An expression cassette favorably consists of the DNA sequences. The expression cassette does not include plasmid elements and reporter sequences. The selection marker can be included in either the expression plasmid/cassette or it can be separately transformed to the host by using co-transformation method. In another embodiment, plasmid elements and reporter sequences are removed from the expression plasmids in order to obtain the expression cassettes for transformation. However, both forms may include and preferably include sequences, which enable locus targeted transformation in the host. The DNA construct may thereby be targeted to a selected locus in the genome of the host.

In another embodiment, the DNA molecule encoding a Dkk1 polypeptide is at least 70% homologous to the DNA sequence of SEQ ID NO: 2. In another embodiment, the DNA molecule encoding a Dkk1 polypeptide is at least 80% homologous to the DNA sequence of SEQ ID NO: 2. In another embodiment, the DNA molecule encoding a Dkk1 polypeptide is at least 85% homologous to the DNA sequence of SEQ ID NO: 2. In another embodiment, the DNA molecule encoding a Dkk1 polypeptide is at least 90% homologous to the DNA sequence of SEQ ID NO: 2. In another embodiment, the DNA molecule encoding a Dkk1 polypeptide is at least 95% homologous to the DNA sequence of SEQ ID NO: 2.

In another embodiment, the DNA molecule encoding a Dkk1 polypeptide is at least 70% homologous to the DNA sequence of SEQ ID NO: 3. In another embodiment, the DNA molecule encoding a Dkk1 polypeptide is at least 80% homologous to the DNA sequence of SEQ ID NO: 3. In another embodiment, the DNA molecule encoding a Dkk1 polypeptide is at least 85% homologous to the DNA sequence of SEQ ID NO: 3. In another embodiment, the DNA molecule encoding a Dkk1 polypeptide is at least 90% homologous to the DNA sequence of SEQ ID NO: 3. In another embodiment, the DNA molecule encoding a Dkk1 polypeptide is at least 95% homologous to the DNA sequence of SEQ ID NO: 3.

In another embodiment, inhibiting hair growth as described herein does not impact the viability of a hair follicle cell. In another embodiment, inhibiting hair growth as described herein does not impact future hair growth. In another embodiment, inhibiting hair growth as described herein does not damage the dermis. In another embodiment, inhibiting hair growth as described herein does not damage the epidermis. In another embodiment, inhibiting hair growth as described herein does not damage the skin. In another embodiment, contacting a Wnt inhibitor of the invention with a hair follicle cell does not impact the viability of a hair follicle cell. In another embodiment, contacting a Wnt inhibitor of the invention with a hair follicle cell does not cause skin damage. In another embodiment, contacting a Wnt inhibitor of the invention with a hair follicle cell does not cause damage to the treated (contacted with a Wnt inhibitor) hair follicle cell.

In one embodiment, provided herein is a method of modulating hair growth in a post-natal subject, comprising the step of inhibiting Wnt/β-catenin signaling in a matured hair follicle cell of said post-natal subject, thereby modulating hair growth in a post-natal subject.

The term “Modulating” refers in one embodiment to transiently inhibiting hair growth.

In another embodiment, the canonical WNT/beta-catenin intercellular signaling pathway is required for initiating the formation of all types of hair follicle placodes during embryogenesis. In another embodiment, the canonical WNT/beta-catenin intercellular signaling pathway is essential post-natally for hair growth. In another embodiment, WNT/beta-catenin signaling initiates hair follicle morphogenesis by direct activation of genes of the tumor necrosis factor (TNF) and TNF receptor families. In another embodiment, DKK1 transiently inhibits the anagen phase of the hair growth cycle. In another embodiment, unexpectedly, expression of Frizzled WNT receptors in developing and post-natal skin is not confined to sites of known activity of the WNT/beta-catenin pathway, suggesting that WNT signaling through alternate pathways contribute to the development and maintenance of the skin and hair follicles. In another embodiment, non-canonical WNT signaling is important for cell movements and polarity in skin epithelia by depleting the function of a key non-canonical WNT signaling pathway component in skin.

In another embodiment, the subject is an animal. In another embodiment, the subject is a mammal. In another embodiment, the subject is a farm animal. In another embodiment, the subject is a pet. In another embodiment, the subject is a human being. In another embodiment, the subject is an adult human being. In another embodiment, the subject is a toddler. In another embodiment, the subject is a post-natal subject. In another embodiment, the subject is a baby. In another embodiment, the subject is a child. In another embodiment, the subject is a senior subject. In another embodiment, a “patient” or a “subject” to be treated by the subject compounds and methods can mean either a human or non-human animal.

In another embodiment, the subject is afflicted with cancer. In another embodiment, the subject is susceptible to keloid formation. In another embodiment, the subject is being prepared for surgery. In another embodiment, the subject is afflicted with odor-causing micro-organisms in hair. In another embodiment, the subject is afflicted with trichiasis. In another embodiment, the subject is afflicted with lice. In another embodiment, the subject is before starting chemotherapy. In another embodiment, the subject utilized the present invention for social and/or sexual reasons related to the social role of hair in human society. In another embodiment, the subject is a male-to-female transsexual. In another embodiment, the subject is afflicted with folliculitis. In another embodiment, the subject is afflicted with hirsutism. In another embodiment, the subject is afflicted with seborrheic dermatitis. In another embodiment, the subject is afflicted with Waardenburg Syndrome. In another embodiment, the subject is afflicted with pseudofolliculitis barbae. In another embodiment, the subject is afflicted with Pseudomonas folliculitis. In another embodiment, the subject is afflicted with psoriasis. In another embodiment, the subject is afflicted with trichilemmal cysts. In another embodiment, the subject is afflicted with seborrheic dermatitis. In another embodiment, the subject is afflicted with dermatitis. In another embodiment, the subject is afflicted with contact dermatitis. In another embodiment, the subject is afflicted with diabetes. In another embodiment, the subject is afflicted with rheumatoid arthritis. In another embodiment, the subject is afflicted with a thyroid disease. In another embodiment, the subject is afflicted with systemic lupus erythematosus. In another embodiment, the subject is afflicted with pernicious anemia. In another embodiment, the subject is afflicted with Addison's disease. In another embodiment, the subject is afflicted with poliosis. In another embodiment, the subject is afflicted with Merkel Cell Carcinoma. In another embodiment, the subject is afflicted with Alopecia Areata.

In another embodiment, a DKK polypeptide provided herein antagonizes the activity of the Wnt pathway. In another embodiment, a DKK polypeptide provided herein agonizes the activity of the Wnt pathway. In another embodiment, a DKK polypeptide provided herein regulates skin and hair growth. In another embodiment, a DKK polypeptide provided herein regulates cells in culture (in vitro), or on cells in a whole animal (in vivo). In another embodiment, a DKK polypeptide provided herein regulates hair follicle cells in culture (in vitro), or on hair follicle cells in a whole animal (in vivo).

In one embodiment, provided herein is a method of modulating hair growth in a post-natal subject, comprising the step of inhibiting Wnt/beta-catenin signaling in a matured hair follicle cell of said post-natal subject with a polypeptide capable of binding to LRP-family, Wnt-associated receptor, thereby modulating hair growth in a post-natal subject.

In one embodiment, the Dickkopf 1 protein (DKK1) blocks Wnt signaling by binding to the LRP Wnt receptor and causing its internalization. In another embodiment, provided herein are strains of transgenic mice carrying two transgenes: K5-rtTA and tetO-Dkk1, where in other embodiments, the first transgene, K5-rtTA, encodes a reverse tet transactivator (rtTA) protein. In another embodiment, the rtTA protein is a transcription factor that is only active in the presence of tetracycline or its relatives such as doxycycline. In other embodiments, in this transgene, rtTA coding sequences are placed under the control of a keratin 5 promoter that directs transcription in basal epidermis and hair follicle outer root sheath cells including the bulge stem cells.

In one embodiment, the second transgene, tetO-Dkk1, encodes the DKK1 Wnt inhibitor protein, under the control of a tetO promoter that is bound and activated by rtTA in the presence of doxycycline. In another embodiment, in mice carrying both transgenes, expression of Dkk1 was induced in epidermal and hair follicle cells by placing the mice on chow containing doxycycline. In another embodiment, untreated and littermate control mice display no evidence of skin or hair follicle abnormalities, whereas in other embodiments, in induced K5-rtTA tetO-Dkk1 mice, hair growth was strongly inhibited.

In one embodiment, histological analysis shows that the hair follicles are arrested in a very early stage of anagen, or in another embodiment, in telogen. In some embodiments, the hair follicles remain in this state during many months of treatment. In another embodiment, during this period the mice lose almost all of their visible hair. In yet another embodiment, the epidermis and dermis of the skin do not display abnormalities detectable by histological analysis or expression of marker genes, where in other embodiments, skin function remains normal.

In one embodiment, upon removal of the drug, hair follicle proliferation and growth resume, indicating that the hair follicles and their associated stem cells are not permanently damaged by the treatment.

In another embodiment, a variety of prokaryotic or eukaryotic cells are used as host-expression systems to express the polypeptides of the present invention. In some embodiments, these include, but are not limited to, microorganisms, such as bacteria transformed with a recombinant bacteriophage DNA, plasmid DNA or cosmid DNA expression vector containing the polypeptide coding sequence; yeast transformed with recombinant yeast expression vectors containing the polypeptide coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the polypeptide coding sequence.

In another embodiment, contacting a matured hair follicle cell with a DKK polypeptide comprises direct delivery of a DKK polypeptide to a matured hair follicle cell. In another embodiment, a DKK polypeptide is delivered to a hair follicle cell in a lotion. In another embodiment, a DKK polypeptide is delivered to a hair follicle cell in a salve. In another embodiment, a DKK polypeptide is delivered to a hair follicle cell in a cream. In another embodiment, a DKK polypeptide is delivered to a hair follicle cell in an ointment. In another embodiment, a DKK polypeptide is delivered to a hair follicle cell in a liposome. In another embodiment, a DKK polypeptide is delivered to a hair follicle cell in a giant micelle.

In some embodiments, non-bacterial expression systems are used (e.g. mammalian expression systems such as CHO cells) to express the polypeptide of the present invention. In another embodiment, the expression vector used to express polynucleotides encoding polypeptides of the present invention in mammalian cells is pCI-DHFR vector comprising a CMV promoter and a neomycin resistance gene.

In some embodiments, in bacterial systems of the present invention, a number of expression vectors can be advantageously selected depending upon the use intended for the polypeptide expressed. In another embodiment, large quantities of polypeptide are desired. In another embodiment, vectors that direct the expression of high levels of the protein product, possibly as a fusion with a hydrophobic signal sequence, which directs the expressed product into the periplasm of the bacteria or the culture medium where the protein product is readily purified are desired. In another embodiment, certain fusion protein engineered with a specific cleavage site to aid in recovery of the polypeptide. In another embodiment, vectors adaptable to such manipulation include, but are not limited to, the pET series of E. coli expression vectors [Studier et al., Methods in Enzymol. 185:60-89 (1990)].

In another embodiment, yeast expression systems are used. In another embodiment, a number of vectors containing constitutive or inducible promoters can be used in yeast as disclosed in U.S. Pat. No. 5,932,447. In another embodiment, vectors which promote integration of foreign DNA sequences into the yeast chromosome are used.

In another embodiment, the expression vector of the present invention can further include additional polynucleotide sequences that allow, for example, the translation of several proteins from a single mRNA such as an internal ribosome entry site (IRES) and sequences for genomic integration of the promoter-chimeric polypeptide.

In another embodiments, mammalian expression vectors comprising a polynucleotide sequence encoding the polypeptides of the invention include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors are useful for in vivo expression of the polypeptides of the present invention since they offer advantages such as lateral infection and targeting specificity. In one embodiment, lateral infection is inherent in the life cycle of, for example, retrovirus and is the process by which a single infected cell produces many progeny virions that bud off and infect neighboring cells. In another embodiment, the result is that a large area becomes rapidly infected, most of which was not initially infected by the original viral particles. In another embodiment, viral vectors are produced that are unable to spread laterally. In another embodiment, this characteristic can be useful if the desired purpose is to introduce a specified Dkk gene into only a localized number of targeted hair follicle cells.

In another embodiment, various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, introduction of nucleic acid by viral infection offers several advantages over other methods such as lipofection and electroporation, since higher transfection efficiency can be obtained due to the infectious nature of viruses.

In another embodiment, it will be appreciated that the polypeptides of the present invention can also be expressed from a nucleic acid construct administered to the individual employing any suitable mode of administration, described hereinabove (i.e., in-vivo gene therapy). In another embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

In another embodiment, plant expression vectors are used. In another embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In another embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In one embodiment, compositions comprising DKK or Dkk1 or nucleotides encoding DKK or small peptidomimetic molecules are used in the methods provided herein for modulating or inhibiting hair growth. Accordingly and in one embodiment, provided herein is a method of inhibiting hair growth in a post-natal subject, comprising the step of contacting a LRP-family, Wnt-associated receptor in a matured hair follicle cell with a DKK polypeptide or its peptidomimetic analog, thereby inhibiting hair growth in a post-natal subject.

In another embodiment, provided herein is a method of inhibiting hair growth in a post-natal subject, comprising the step of contacting a matured hair follicle cell with a DNA construct comprising the nucleic acid sequence encoding a DKK polypeptide, thereby inhibiting hair growth in a post-natal subject.

In one embodiment, provided herein is a method of modulating hair growth in a post-natal subject, comprising the step of contacting a LRP-family, Wnt-associated receptor in a matured hair follicle cell with a DKK polypeptide or its peptidomimetic analog, thereby inhibiting hair growth in a post-natal subject.

In another embodiment, provided herein is a method of modulating hair growth in a post-natal subject, comprising the step of contacting a matured hair follicle cell with a DNA construct comprising the nucleic acid sequence encoding a DKK polypeptide, thereby inhibiting hair growth in a post-natal subject.

In another embodiment, provided herein is a method of modulating hair growth in a post-natal subject, comprising the step of inhibiting Wnt/beta-catenin signaling in a matured hair follicle cell of said post-natal subject, thereby modulating hair growth in a post-natal subject.

In one embodiment, provided herein is a method of temporarily removing hair on a subject, comprising the step of contacting skin of the subject with a topical composition comprising DKK polypeptide or its peptidomimetic, thereby blocking LRP-family, Wnt-associated receptor in a mature hair follicle cell in the skin, thus temporarily removing hair on a subject.

In another embodiment, provided herein is a method of temporarily inhibiting hair growth on a subject, comprising the step of contacting skin of the subject with a topical composition comprising DKK polypeptide or its peptidomimetic, thereby blocking LRP-family, Wnt-associated receptor in a mature hair follicle cell in the skin, thus temporarily removing hair on a subject.

Various methods, in some embodiments, can be used to introduce the expression vector of the present invention into the host cell system. In some embodiments, such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In some embodiments, transformed cells are cultured under effective conditions, which allow for the expression of high amounts of recombinant polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

In some embodiments, depending on the vector and host system used for production, resultant polypeptides of the present invention either remain within the recombinant cell, secreted into the fermentation medium, secreted into a space between two cellular membranes, such as the periplasmic space in E. coli; or retained on the outer surface of a cell or viral membrane.

In another embodiment, following a predetermined time in culture, recovery of the recombinant polypeptide is effected.

In one embodiment, the phrase “recovering the recombinant polypeptide” used herein refers to collecting the whole fermentation medium containing the polypeptide and need not imply additional steps of separation or purification.

In another embodiment, polypeptides of the present invention are purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

In another embodiment, to facilitate recovery, the expressed coding sequence can be engineered to encode the polypeptide of the present invention and fused cleavable moiety. In one embodiment, a fusion protein can be designed so that the polypeptide can be readily isolated by affinity chromatography; e.g., by immobilization on a column specific for the cleavable moiety. In one embodiment, a cleavage site is engineered between the polypeptide and the cleavable moiety and the polypeptide can be released from the chromatographic column by treatment with an appropriate enzyme or agent that specifically cleaves the fusion protein at this site [e.g., see Booth et al., Immunol. Lett. 19:65-70 (1988); and Gardella et al., J. Biol. Chem. 265:15854-15859 (1990)].

In another embodiment, the polypeptide of the present invention is retrieved in “substantially pure” form.

In another embodiment, the phrase “substantially pure” refers to a purity that allows for the effective use of the protein in the applications described herein.

In another embodiment, the polypeptide of the present invention can also be synthesized using in vitro expression systems. In another embodiment, in vitro synthesis methods are well known in the art and the components of the system are commercially available.

In some embodiments, the recombinant polypeptides are synthesized and purified; their therapeutic efficacy can be assayed either in vivo or in vitro.

### Pharmaceutical Compositions and Methods of Administration

In another aspect, the present invention provides pharmaceutical preparations comprising, a DKK polypeptide, a Wnt/beta-catenin signaling inhibitor, or a compound that blocks LRP-family, Wnt-associated receptor, formulated in an amount sufficient to regulate, in vivo, Wnt pathway, e.g., proliferation or other biological consequences of mis-expression of Wnt.

DKK1 of the present invention and pharmaceutical compositions comprising same can be, in another embodiment, administered to a subject by any method known to a person skilled in the art, such as parenterally, paracancerally, transmucosally, transdermally, intramuscularly, intravenously, intra-dermally, subcutaneously, intra-peritonealy, intra-ventricularly, intra-cranially, intra-vaginally or intra-tumorally.

In another embodiment of methods and compositions of the present invention, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulsions, oils and the like. In another embodiment of the present invention, the active ingredient is formulated in a capsule. In accordance with this embodiment, the compositions of the present invention comprise, in addition to the active compound (e.g. the mimetic compound, peptide or nucleotide molecule) and the inert carrier or diluent, a hard gelating capsule.

In another embodiment, the pharmaceutical compositions are administered by intravenous, intra-arterial, or intra-muscular injection of a liquid preparation. Suitable liquid formulations include solutions, suspensions, dispersions, emulsions, oils and the like. In another embodiment, the pharmaceutical compositions are administered intravenously and are thus formulated in a form suitable for intravenous administration. In another embodiment, the pharmaceutical compositions are administered intra-arterially and are thus formulated in a form suitable for intra-arterial administration. In another embodiment, the pharmaceutical compositions are administered intra-muscularly and are thus formulated in a form suitable for intra-muscular administration.

In another embodiment, the pharmaceutical compositions are administered topically to body surfaces and are thus formulated in a form suitable for topical administration. Topical formulations include, in another embodiment, gels, ointments, creams, lotions, drops and the like.

Accordingly and in one embodiment, provided herein is a method of temporarily removing hair on a subject, comprising the step of contacting skin of the subject with a topical composition comprising DKK polypeptide or its peptidomimetic, thereby blocking LRP-family, Wnt-associated receptor in a mature hair follicle cell in the skin, thus temporarily removing hair on a subject.

Suitable topical vehicles for use with the formulations of the invention are well known in the cosmetic and pharmaceutical arts, and include such vehicles (or vehicle components) as water; organic solvents such as alcohols (particularly lower alcohols readily capable of evaporating from the skin such as ethanol), glycols (such as glycerin), aliphatic alcohols (such as lanolin); mixtures of water and organic solvents (such as water and alcohol), and mixtures of organic solvents such as alcohol and glycerin (optionally also with water); lipid-based materials such as fatty acids, acylglycerols (including oils, such as mineral oil, and fats of natural or synthetic origin), phosphoglycerides, sphingolipids and waxes; protein-based materials such as collagen and gelatin; silicone-based materials (both non-volatile and volatile) such as cyclomethicone, demethiconol and dimethicone copolyol (Dow Corning); hydrocarbon-based materials such as petrolatum and squalane; anionic, cationic and amphoteric surfactants and soaps; sustained-release vehicles such as microsponges and polymer matrices; stabilizing and suspending agents; emulsifying agents; and other vehicles and vehicle components that are suitable for administration to the skin, as well as mixtures of topical vehicle components as identified above or otherwise known to the art. The vehicle may further include components adapted to improve the stability or effectiveness of the applied formulation, such as preservatives, antioxidants, skin penetration enhancers, sustained release materials, and the like. Examples of such vehicles and vehicle components are well known in the art and are described in such reference works as Martindale—The Extra Pharmacopoeia (Pharmaceutical Press, London 1993) and Martin (ed.), Remington's Pharmaceutical Sciences.

The choice of a suitable vehicle will depend on the particular physical form and mode of delivery that the formulation is to achieve. Examples of suitable forms include liquids (including dissolved forms of the cations of the invention as well as suspensions, emulsions and the like); solids and semisolids such as gels, foams, pastes, creams, ointments, “sticks” (as in lipsticks or underarm deodorant sticks), powders and the like; formulations containing liposomes or other delivery vesicles; rectal or vaginal suppositories, creams, foams, gels or ointments; and other forms. Typical modes of delivery include application using the fingers; application using a physical applicator such as a cloth, tissue, swab, stick or brush (as achieved for example by soaking the applicator with the formulation just prior to application, or by applying or adhering a prepared applicator already containing the formulation—such as a treated or premoistened bandage, wipe, washcloth or stick—to the skin); spraying (including mist, aerosol or foam spraying); dropper application (as for example with ear drops); sprinkling (as with a suitable powder form of the formulation); and soaking.

Methodologies and materials for preparing formulations in a variety of forms are also described in Anthony L. L. Hunting (ed.), “A Formulary of Cosmetic Preparations (Vol. 2)—Creams, Lotions and Milks,” Micelle Press (England, N.J. 1993). See, for example, Chapter 7, pp. 5-14 (oils and gels); Chapter 8, pp. 15-98 (bases and emulsions); Chapter 9, pp. 101-120 (“all-purpose products”); Chapter 10, pp. 121-184 (cleansing masks, creams, lotions); Chapter 11, pp. 185-208 (foundation, vanishing and day creams); Chapter 12, pp. 209-254 (emollients); Chapter 13, pp. 297-324 (facial treatment products); Chapter 14, pp. 325-380 (hand products); Chapter 15, pp. 381-460 (body and skin creams and lotions); and Chapter 16, pp. 461-484 (baby products); the contents of which are incorporated herein by reference.

In another embodiment, the pharmaceutical composition is administered as a suppository, for example a rectal suppository or a urethral suppository. In another embodiment, the pharmaceutical composition is administered by subcutaneous implantation of a pellet. In another embodiment, the pellet provides for controlled release of active agent over a period of time.

In another embodiment, the active compound is delivered in a vesicle, e.g. a liposome.

In other embodiments, carriers or diluents used in methods of the present invention include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

In other embodiments, pharmaceutically acceptable carriers for liquid formulations are aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of animal, vegetable, or synthetic origin, for example, peanut oil, soybean oil, olive oil, sunflower oil, fish-liver oil, another marine oil, or a lipid from milk or eggs.

In another embodiment, parenteral vehicles (for subcutaneous, intravenous, intra-arterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of animal, vegetable, or synthetic origin, for example, peanut oil, soybean oil, olive oil, sunflower oil, fish-liver oil, another marine oil, or a lipid from milk or eggs.

In other embodiments, the compositions further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI, acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylmethyl cellulose), viscosity increasing agents (e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxamines), coating and film forming agents (e.g. ethyl cellulose, acrylates, polymethacrylates) and/or adjuvants. Each of the above excipients represents a separate embodiment of the present invention.

In another embodiment, the pharmaceutical compositions provided herein are controlled-release compositions, i.e. compositions in which the active compound is released over a period of time after administration. Controlled- or sustained-release compositions include formulation in lipophilic depots (e.g. fatty acids, waxes, oils). In another embodiment, the composition is an immediate-release composition, i.e. a composition in which of the active compound is released immediately after administration.

In another embodiment, the pharmaceutical composition is delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials are used; e.g. in microspheres in or an implant. In yet another embodiment, a controlled release system is placed in proximity to the target cell, thus requiring only a fraction of the systemic dose (see, e.g., Goodson, in Medical Applications of Controlled Release, supra, vol. 2, pp. 115-138 (1984); and Langer R, Science 249: 1527-1533 (1990).

The compositions also include, in another embodiment, incorporation of the active material into or onto particulate preparations of polymeric compounds such as polylactic acid, polglycolic acid, hydrogels, etc, or onto liposomes, microemulsions, micelles, unilamellar or multilamellar vesicles, erythrocyte ghosts, or spheroplasts.) Such compositions will influence the physical state, solubility, stability, rate of in vivo release, and rate of in vivo clearance.

Also included in the present invention are particulate compositions coated with polymers (e.g. poloxamers or poloxamines) and the compound coupled to antibodies directed against tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

Each of the above additives, excipients, formulations and methods of administration represents a separate embodiment of the present invention.

In one embodiment, the methods of the present invention comprise administering an active compound as the sole active ingredient. However, also encompassed within the scope of the present invention are methods for treating diseases and disorders that comprise administering the active compound in combination with one or more therapeutic agents. These agents include, but are not limited to, chemotherapeutic agents. In another embodiment, these agents are appropriate for the disease or disorder that is being treated, as is well known in the art.

In one embodiment, the methods of the present invention comprise administering an active compound as the sole active ingredient. However, also encompassed within the scope of the present invention are methods for treating diseases and disorders that comprise administering the active compound in combination with one or more therapeutic agents.

All literature references cited in the present specification are hereby incorporated by reference in their entirety.

The following examples are presented in order to more fully illustrate the preferred embodiments of the invention. They should in no way be construed, however, as limiting the broad scope of the invention.

### EXAMPLES

Wnt/β-catenin signaling is activated in hair follicles at anagen onset, and localizes to proliferating matrix cells and differentiating hair shaft precursor cells. To determine whether Wnt/β-catenin signaling is required for hair follicle cyclical growth in postnatal life, we used K5-rtTA tetO-Dkk1 bi-transgenic mice in which expression of the secreted Wnt inhibitor Dickkopf1 (DKK1) can be induced in the hair follicle outer root sheath including stem cells, by dosage with oral doxycycline. Induction of Dkk1 expression in mid-anagen prevented nuclear localization of β-catenin in epithelial matrix cells but not in the dermal papilla, and caused cessation of proliferation and premature entry into catagen. To determine whether Dkk1 also blocks anagen onset, Dkk1 expression was induced in telogen, and hair shafts were plucked to initiate a new growth cycle. Unexpectedly, follicles entered very early anagen, but epithelial proliferation and cyclin D1 expression, and BMP signaling, which is required for hair follicle differentiation, were inhibited and the follicles did not express differentiation markers or progress through subsequent stages of anagen. However, follicular structures including the dermal papilla, and expression of the bulge stem cell marker K15 were maintained even over long periods of Dkk1 expression. To determine whether the effects of Dkk1 were reversible, mice were maintained on doxycycline for up to 15 months, followed by removal of doxycycline. Remarkably, hair follicles showed normal proliferation and differentiation following doxycycline withdrawal, with or without hair plucking to initiate a new growth cycle. Spontaneous hair re-growth following doxycycline removal suggested that the follicles were primed for hair re-growth rather than being maintained in a true telogen phase. These data clearly shows Wnt inhibition as a promising method to reversibly inhibit growth of unwanted hair.

### Experimental Details

### Transgenic Mice Inducibly Expressing Dkk1

To study the effects of ectopic Dkk1 on skin, double transgenic K5-rtTA; tetO-Dkk1 mice were generated as described previously (Chu et al., 2004, Development 131, 4819-4829). Mice were genotyped by PCR analysis of tail biopsy. For doxycycline-inducible expression of the Dkk1 transgene, mice carrying the K5-rtTA and the tetO-Dkk1 transgenes were fed chow containing 6 g/kg doxycycline (BioServ Inc.). In experiments studying the effects of Dkk1 expression on hair follicle development and hair growth before weaning, the nursing mother was placed on doxycycline chow. For depilation experiments, hair was clipped and the skin was treated with Neet hair remover cream (Reckitt & Colman Inc.) for 6 min. Neet cream was washed off with warm water and the mice were wiped dry. The Institutional Animal Care and Use Committee of the University of Pennsylvania approved all experimental procedures involving mice.

### Histology and In Situ Hybridization

For histological analysis, skin samples were fixed in 4% paraformaldehyde overnight at 4° C. and embedded in paraffin. Tissue blocks were sectioned at 5 μM and stained with hematoxylin and eosin. In situ hybridization using digoxigenin-labeled probes was carried out according to published protocols (Millar et al., 1995, Development 121, 3223-3232; Millar et al., 1999, Dev Biol 207, 133-149). The probe for Dkk1 has been described previously (Andl et al., 2002, Dev Cell 2, 643-653).

### Immunohistochemistry and Immunofluorescence

Skin sections were baked for 30 min at 55° C., and then deparaffinized. For immunohistochemistry, heat-induced antigen retrieval was performed by microwaving sections in 10 mM sodium citrate, pH 6.0 for 6 min. Endogenous peroxidase activity was quenched by incubating the sections in 0.6% hydrogen peroxide in methanol for 15 min. All incubations were performed at room temperature unless otherwise stated. Primary antibodies were applied (β-catenin, 1:1000, Sigma; cyclin D1, 1:25, Cell Signaling Technology) and the sections were incubated overnight at 4° C. Subsequently, sections were incubated with biotinylated secondary antibodies (Vector Laboratories) for 30 min. Visualization of the bound biotin was the performed using Vectastain Elite ABC kit (Vector Laboratories) followed by DAB substrate kit according to manufacture's protocol (Vector Laboratories). The following primary antibodies and dilutions were used for immunofluorescence: AE13 at 1:100 (Abcam); BMP-6 at 1:50 (Santa Cruz Biotechnology); GATA-3 at 1:40 (Santa Cruz Biotechnology); cytokeratin 15 at 1:50 (Vector Laboratories); Ki67 at 1:40 (NovoCastra); p63 at 1:100 (Thermo Scientific) and phospho-Smad1/5/8 at 1:50 (Cell Signaling Technology). Antigen retrieval was performed either in Tris-EDTA buffer (pH 8.0) for 8 min or 10 mM sodium citrate (pH 6.0) for 10 min. Sections were incubated with primary antibodies overnight at 4° C., followed by 30-min incubation with biotinylated secondary antibodies. The resulting complex was visualized with Fluorescein- or Texas Red-conjugated streptavidin (Vector Laboratories). The slides were mounted with VECTASHIELD Mounting Medium with DAPI (Vector Laboratories), and the immunofluorescence was viewed under a LEICA DM4000B microscope (Leica Microsystems). Images were captured by using LEICA DC500 digital camera and Leica FireCam software version 1.4 (Leica Microsystems).

### Proliferation and Apoptosis Measurements

Proliferation was measured by intraperitoneal injection of BrdU (Roche, 2.5 mg/ml in PBS; 50 μg BrdU/g body weight) 1 hour before sacrificing the animals. BrdU incorporation was detected by immunofluorescence on 5 μm-thick paraffin sections using BrdU detection Kit II (Roche). To quantify BrdU positive cells, digital photographs of the BrdU-labeled hair bulbs were analyzed. The total number of BrdU-labeled cells/the total number of DAPI-labeled cells was calculated. For the detection of apoptotic cells, the TUNEL enzyme kit was used according to the manufacture's instructions (Roche).

### Example 1

### Ectopic Expression of Dkk1 During the Growth Phase of the Embryonic Hair Cycle Inhibits Proliferation and Causes Rapid Regression of Hair Follicles

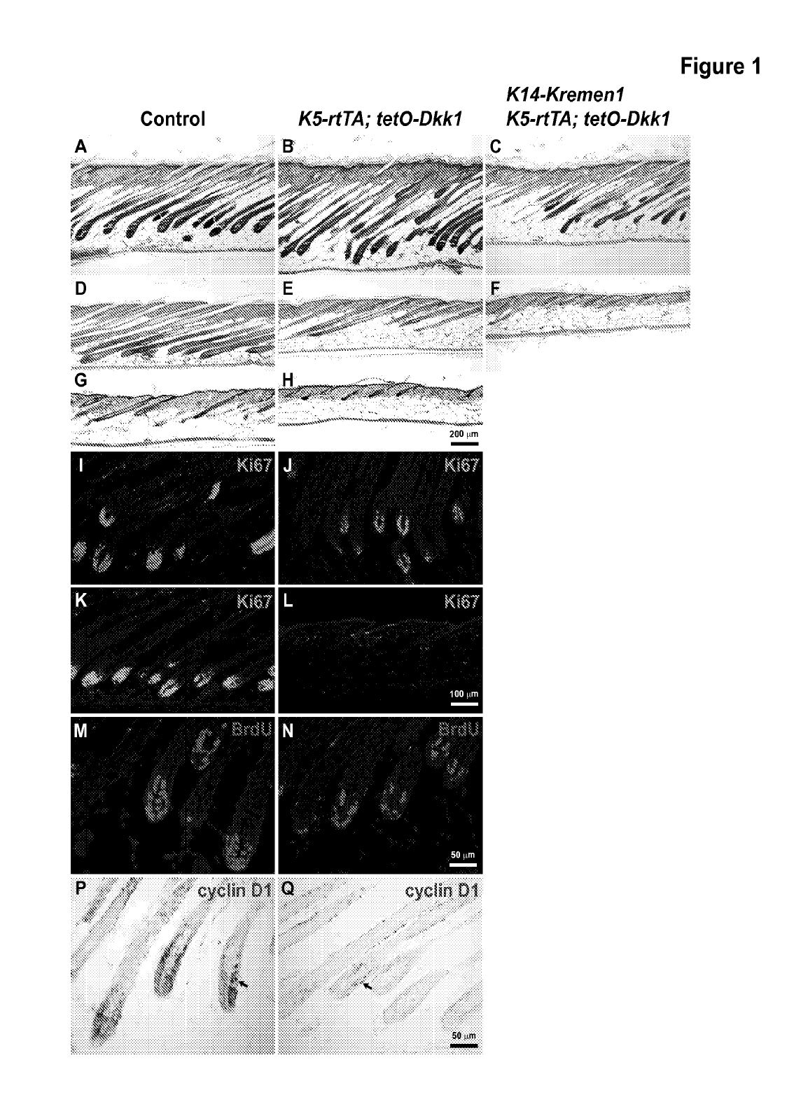

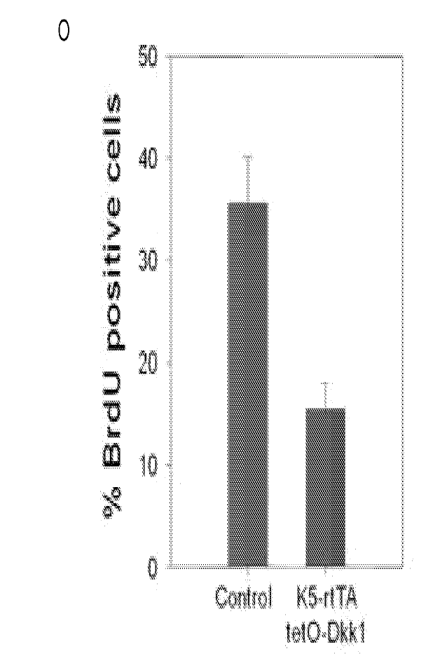

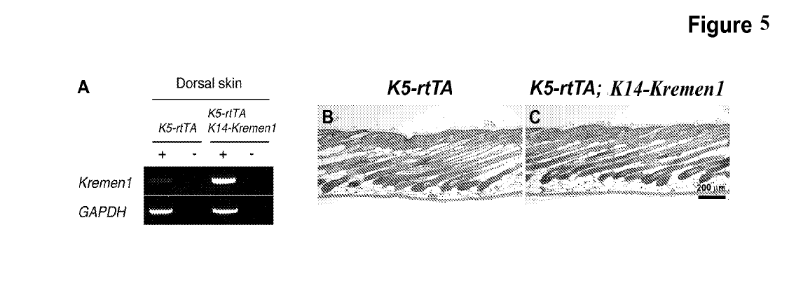

We first examined the effects of ectopic expression of the Wnt inhibitor Dkk1 during the growth phase of the embryonic hair cycle. Expression of Dkk1 was induced at postnatal day 4, and dorsal skin was harvested at postnatal days 8, 14, and 20. All K5-rtTA; tetO-Dkk1 double transgenic skin samples showed rapid regression of hair follicles, compared with controls (FIGS. 1A-1B, 1D-1E, and 1G-1H). Kremen1 is required for inhibition of Wnt signaling by Dkk1. While endogenous Kremen1 is expressed in the skin, variations in its levels during the hair growth cycle might affect Dkk1's inhibitory activity. We therefore also examined the effects of ectopic Dkk1 expression in K5-rtTA; tetO-Dkk1 triple transgenic mice that additionally carry a keratin 14 promoter-driven Kremen1 transgene. The dorsal skin of K14-Kremen1 transgenic mice showed increased expression of Kremen1 compared with controls (FIG. 5A). However, ectopic expression of Kremen1 alone resulted in normal hair follicle growth in the absence of ectopic Dkk1 (FIG. 5B-5C). In contrast, ectopic co-expression of Dkk1 and Kremen1 in K14-Kremen1; K5-rtTA; tetO-Dkk1 triple transgenic mice led to faster regression of hair follicles than that observed in K5-rtTA; tetO-Dkk1 double transgenic mice (FIGS. 1B-1C and 1E-1F).

To determine what caused the rapid regression of transgenic hair follicles, we assayed for changes in apoptosis and proliferation. No alterations in the percentages of apoptotic cells in Dkk1 expressing hair follicles were detected compared to controls. We examined proliferation both by staining for the proliferation antigen Ki67 and by assaying for BrdU incorporation. Transgenic hair follicles showed greatly reduced proliferation, compared with controls (FIGS. 1I-1N). Quantitation of BrdU positive cells showed that only 15% of nuclei are positive in Dkk1 expressing hair bulbs, compared with 35% in controls after 4 days of induction from postnatal day 4 (FIG. 10). Cyclin D1 is a direct Wnt/p-catenin target gene and an important regulator of cellular proliferation that helps initiate transition from the late G1 phase to the S phase of cell cycle (Alonso and Fuchs, 2003, Genes Dev 17, 1189-1200; Tetsu and McCormick, 1999, Nature 398, 422-426). Transgenic hair follicles showed greatly diminished expression of cyclin D1 (FIG. 1Q). These results show that decreased cyclin D1 expression can contribute to the observed effects of Dkk1 on hair follicle proliferation.

### Example 2

### Induced Ectopic Expression of Dkk1 does not Affect Maintenance of Hair Follicle Stem Cells

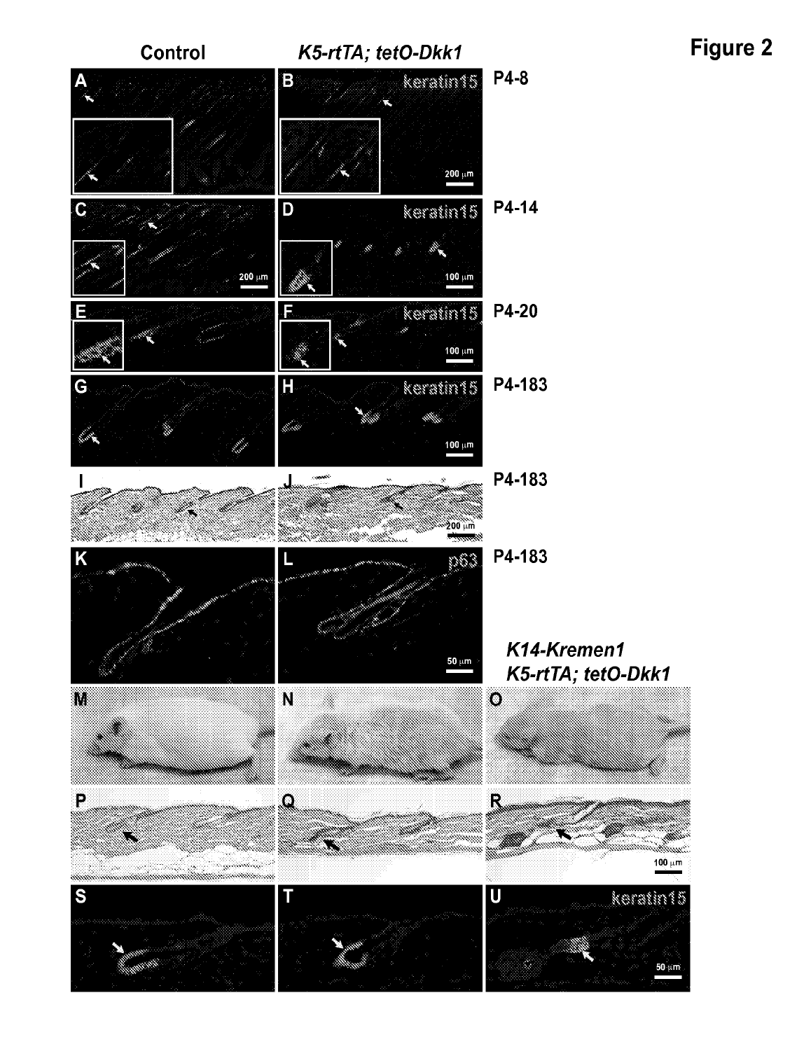

To ask whether maintenance of hair follicle stem cells is affected by Wnt inhibition we used an antibody to keratin (K) 15, a specific marker for epithelial stem cells in the hair follicle bulge (Liu et al., 2003, J Invest Dermatol 121, 963-968). In contrast to the dramatic effects of Dkk1 expression on proliferation, expression of K15 was readily detected in the transgenic hair follicles (FIGS. 2B, 2D, and 2F). To determine whether hair follicles and their stem cells persist during long-term Wnt inhibition, we induced Dkk1 expression from postnatal day 4, and examined skin histology and expression of the K15 stem cell marker and p63 basal cell marker after 6 months. We found that K15 expression was similar in control and Dkk1 expressing hair follicles (FIGS. 2G-2H), and hair follicle structures were maintained during these long periods of Dkk1 expression (FIG. 2J). No difference in the expression of p63 was observed between control and Dkk1 expressing skin (FIGS. 2K-2L). K14-Kremen1; K5-rtTA; tetO-Dkk1 triple transgenic mice displayed increased loss of external hair (FIG. 20) and abnormal hair follicle morphology (FIG. 2R) compared to the K5-rtTA; tetO-Dkk1 double transgenic mice and controls (FIGS. 2M-2M and 2P-2Q). However, K14-Kremen1; K5-rtTA; tetO-Dkk1 triple transgenic skin showed persistence of hair follicle stem cells (FIG. 2U). These results demonstrate that hair follicle stem cells are maintained even during long-term Wnt inhibition.

### Example 3

### The Plucking-Induced Anagen Phase is Blocked by Ectopic Dkk1

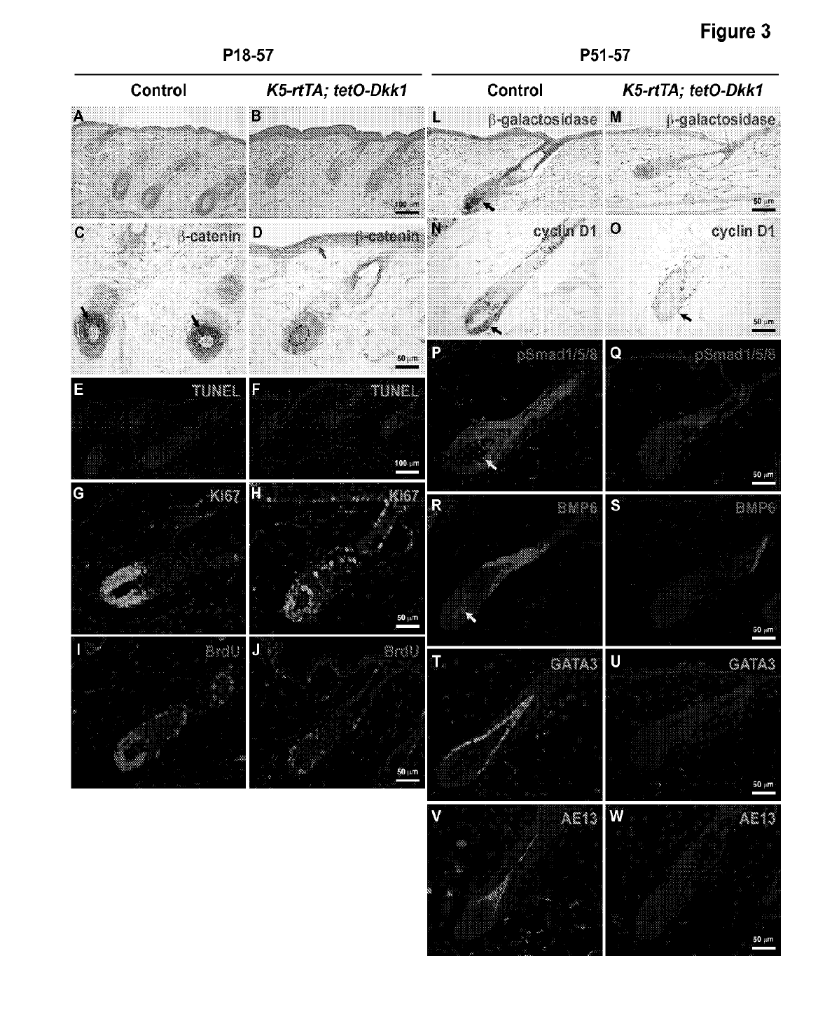

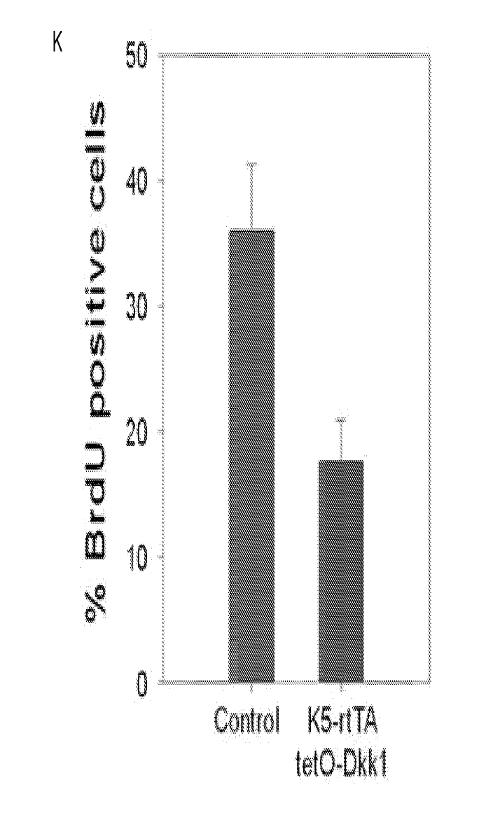

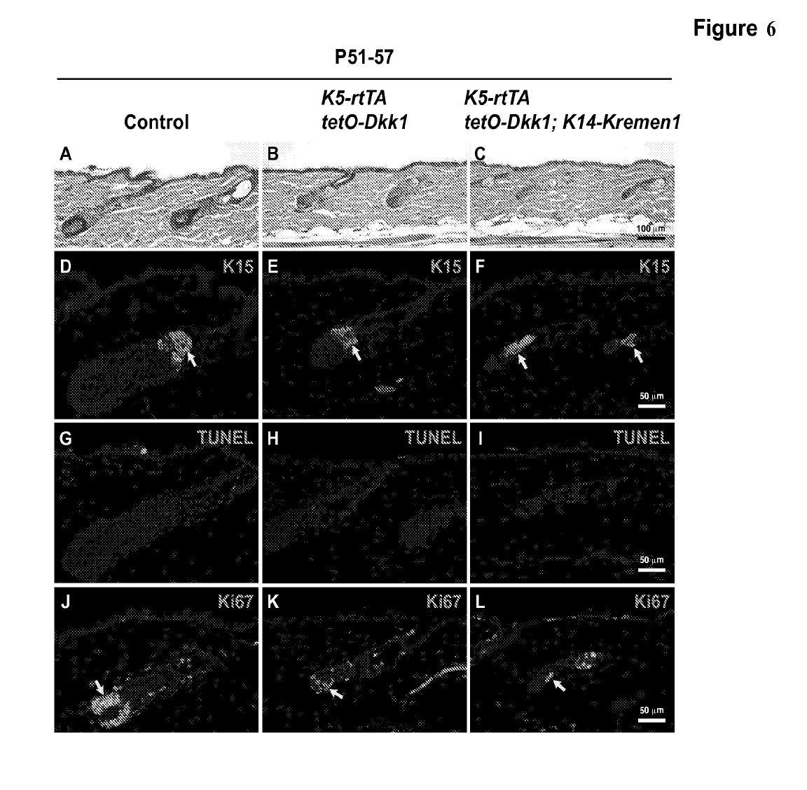

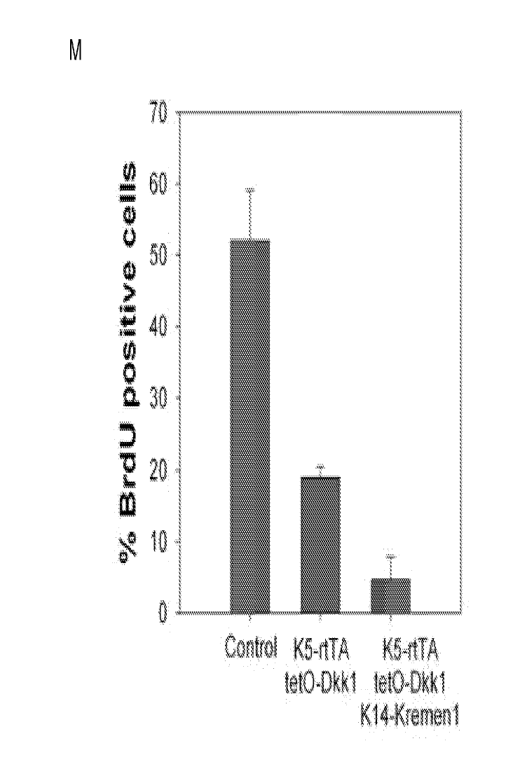

We next asked whether ectopic Dkk1 expression could block onset of the anagen growth phase in adult hair follicles. Two animal groups were used for these experiments. For both groups, we began doxycycline induction of Dkk1 expression during telogen, either at postnatal day 18 or at postnatal day 51. For the P18-57 group, hair plucking was performed at postnatal day 52 to induce a new anagen growth phase, and skin histology was examined 5 days after depilation. For the P51-57 group, hair plucking was carried out on postnatal day 54, and dorsal skin was harvested at postnatal day 57, 3 days after depilation. In both cases, entry into the anagen growth phase was inhibited in Dkk1 expressing skin compared with controls (FIGS. 3A-3B and 3L-3M). K14-Kremen1; K5-rtTA; tetO-Dkk1 triple transgenic mice also showed failure of onset of the anagen growth phase (FIG. 6C). However, expression of the stem cell marker keratin 15 was maintained even in K14-Kremen1; K5-rtTA; tetO-Dkk1 triple transgenic plucked hair follicles (FIG. 6F).

To confirm inhibition of Wnt signaling in plucked Dkk1 expressing hair follicles, we used immunohistochemistry for β-catenin. Nuclear localized β-catenin was clearly present in the matrix of control hair follicles 5 days after hair plucking (FIG. 3C, black arrows), and, at slightly lower levels, in the dermal papilla. Nuclear β-catenin was dramatically reduced in Dkk1 expressing matrix cells, although low levels of nuclear β-catenin were still detectable in the dermal papilla (FIG. 3D). Expression of β-catenin at the cell membrane was unaffected by Dkk1 expression (FIG. 3D, blue arrow). We also examined activity of the Wnt signaling pathway using Conductin-lacZ Wnt reporter mice. β-galactosidase expression, indicating Wnt signaling activity, was readily detected in control hair follicle bulbs (FIG. 3L, black arrow), but was not observed in Dkk1 expressing hair follicles (FIG. 3M). Expression of the direct Wnt/β-catenin target gene and cell proliferation regulator cyclin D1, was also significantly reduced in Dkk1 expressing follicles (FIG. 30, black arrow).

To examine the basis of failure of the anagen growth phase in adult Dkk1 expressing skin, we used the TUNEL assay to measure apoptosis, and assayed for proliferation by immunofluorescence for Ki67 and BrdU. Dkk1 expressing hair follicles did not exhibit increased apoptosis compared with controls (FIGS. 3E and 3F). K14-Kremen1; K5-rtTA; tetO-Dkk1 triple transgenic hair follicles also showed no increase in the number of TUNEL-positive cells (FIG. 61). However, cell proliferation was substantially reduced, although not entirely absent, in K5-rtTA; tetO-Dkk1 double transgenic (FIGS. 3H and 3J) and in K14-Kremen1; K5-rtTA; tetO-Dkk1 triple transgenic hair follicles (FIG. 6L). Approximately 37% of cells in control hair bulbs were positive for BrdU, while in double transgenic hair follicle bulbs this number was decreased to approximately 18% (FIG. 3K). Only 4.5% of nuclei were positive for BrdU in K14-Kremen1; K5-rtTA; tetO-Dkk1 triple transgenic hair bulbs (FIG. 6M).

### Example 4

### BMP Signaling Pathway Activity is Blocked in Dkk1-Expressing Hair Follicles

BMP signaling is essential for differentiation of the hair shaft and inner root sheath (Andl et al., 2004, Development 131, 2257-2268). To examine the effects of Wnt inhibition on activity of the BMP signaling pathway, we carried out immunofluorescence for phosphorylated Smad1/5/8 (pSmad1/5/8). Cells responding to BMP signals can be identified by the presence of pSmad1/5/8 in the nucleus (von Bubnoff and Cho, 2001, Dev Biol 239, 1-14). As judged by immunostaining for pSmad1/5/8, BMP signaling was active in the dermal papilla and epithelial cells of control hair follicles (FIG. 3P, yellow arrow), while Dkk1-expressing hair follicles did not display pSmad1/5/8 nuclear staining. These data indicate that BMP signaling in the plucking-induced anagen phase is blocked by Wnt inhibition (FIG. 3Q). BMP6 has been reported to play an important role in hair growth (Rendl et al., 2008, Genes Dev 22, 543-557). Interestingly and surprisingly, we found that the BMP6 protein expression in the dermal papilla of plucked hair follicles is blocked by Wnt inhibition (FIG. 3S). As expression of Dkk1 in the hair follicle epithelium does not appear to affect nuclear localization of β-catenin in the dermal papilla, the effects of ectopic Dkk1 on dermal BMP6 expression may be indirect. Consistent with absence of BMP signaling activity, Dkk1-expressing hair follicles did not express GATA3, a marker for the inner root sheath (IRS) (FIG. 3U) or acidic hair keratin AE13, a marker for hair shaft differentiation (FIG. 3W).

### Example 5

### The Effects of Long-Term Dkk1 Expression in Inhibiting Hair Growth are Reversible Following Removal of the Inhibitor

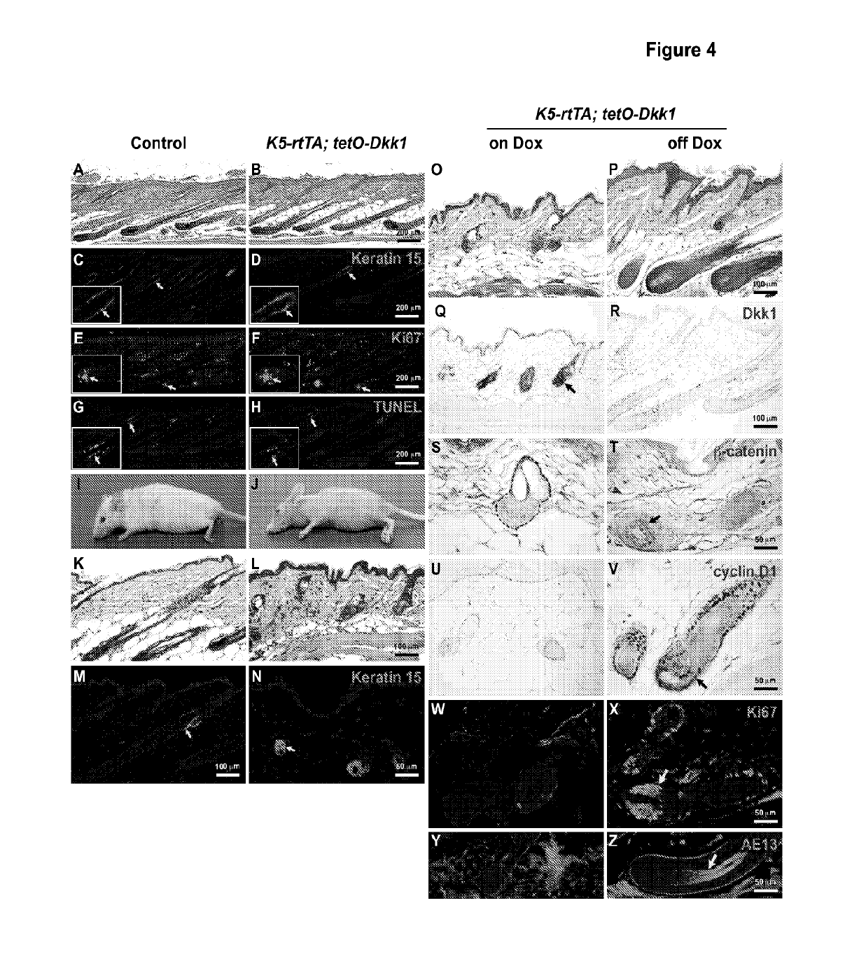

Since the stem cell marker keratin 15 is still expressed even during long-term Wnt inhibition (FIG. 2H), we next asked whether the block to hair growth is reversed following removal of the inhibitor. Control and double transgenic mice were maintained on doxycycline for 14.5 months with a 1-month break in the 10^{th} month. At 14.5 months the mice were removed from doxycycline food and hair was plucked to initiate a new cycle of hair follicle growth. Mice were sacrificed 21 days after hair plucking, and skin histology was assayed. As shown in FIGS. 4A and 4B, following removal of doxycycline and hair plucking, a new cycle of hair growth was induced in double transgenic as well as control skin. Expression of the keratin 15 stem cell marker was maintained in both control and experimental animals (FIGS. 4C and 4D, yellow arrows), and proliferation and apoptosis levels were similar in both cases (FIGS. 4E-4H, yellow arrows). These results demonstrate that the effects of Dkk1 in inhibiting hair growth are reversible.

To determine whether the effects of very long-term Dkk1 expression are reversible, mice were maintained on doxycycline from postnatal day 21 until 15.5 months of age. By this stage the experimental mice completely lacked external hair (FIG. 4J). Histological examination and immunofluorescence for keratin 15 in double transgenic mice revealed persistence of hair follicle structures (FIG. 4L) and their associated stem cells (FIG. 4N), although hair follicle morphology was frequently abnormal. Surprisingly, after 14 days of doxycycline withdrawal, regrowth of numerous follicles occurred spontaneously in the skin of double transgenic mice (FIG. 4P). These data indicate persistence of functional hair follicle stem cells and reversibility of the effects of Wnt inhibition even following very long-term Dkk1 induction. These data further indicate that the Dkk1-inhibited hair follicles are primed for hair regrowth rather than being maintained in a normal telogen resting phase. Consistent with this, Dkk1-inhibited hair follicles have a morphology consistent with arrest in very early anagen, rather than a true telogen phase.

To confirm loss of Dkk1 expression following removal of doxycycline, we carried out in situ hybridization for Dkk1 on skin biopsies taken before and 14 days after removal of doxycycline chow. Dkk1 expression was clearly detected in the epidermis and hair follicles from double transgenic mice on doxycycline chow (FIG. 4Q, black arrow), and was absent or very much reduced following 14 days off doxycycline (FIG. 4R). We next asked whether Wnt signaling resumed in hair follicles following removal of doxycycline, by immunostaining for nuclear β-catenin. Nuclear β-catenin was not observed in hair follicles from double transgenic mice on doxycycline (FIG. 4S), but was clearly detectable in the hair follicle matrix following doxycycline withdrawal (FIG. 4T, black arrow). As expected from the morphology of the hair follicles, strong expression of cyclin D1 was observed following doxycycline withdrawal (FIG. 4V, black arrow) and proliferation was dramatically enhanced (FIG. 4X, yellow arrow). In contrast with hair follicles lacking acidic hair keratin AE13 expression in experimental mice on doxycycline (FIG. 4Y), AE13 was present in the hair shaft of the newly forming hair follicles following doxycycline withdrawal (FIG. 4Z, yellow arrow). Therefore, the effects of Dkk1 on β-catenin nuclear localization, cyclin D1 expression, proliferation, and differentiation were reversed by removal of doxycycline even following very long-term ectopic expression of Dkk1.

In summary, our data show that Wnt signaling is required throughout anagen to maintain proliferation of hair follicle matrix cells and hair growth. Consistent with lack of expression of nuclear β-catenin or Wnt reporter transgenes in resting hair follicles, Wnt inhibition does not affect the maintenance of hair follicle stem cells or hair follicle structures. These data are in contrast to the effects of deletion of epithelial β-catenin. These observations show that β-catenin may perform additional, non-Wnt-dependent roles in maintenance of the hair follicle stem cell compartment. These may be related to β-catenin's adhesive functions or its participation in another signaling pathway. Alternatively, extremely low levels of Wnt/β-catenin signaling, not detectable by the nuclear β-catenin or reporter gene assays, persisting in resting hair follicles, may be required for stem cell maintenance, and are removed by deletion of the β-catenin gene but not by the actions of secreted Dkk1, even in the presence of forced expression of Kremen. In either case, our data show that inhibition of Wnt signaling by Dkk1 effectively blocks hair growth without removing hair follicle stem cells or destroying hair follicle structures, even over very long periods of inhibition. These results show Wnt inhibition as a promising method to reversibly inhibit growth of unwanted hair.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method of modulating or reversibly inhibiting hair growth in a post-natal subject without damaging skin or damaging a hair follicle, comprising the step of contacting a LRP-family, Wnt-associated receptor in a matured hair follicle cell with a DKK polypeptide, its peptidomimetic analog or a DNA construct comprising a nucleic acid sequence encoding said DKK polypeptide, thereby reversibly inhibiting hair growth in a post-natal subject.

2. (canceled)

3. The method of claim 1, whereby the hair follicle cell comprises a hair shaft, a dermal papilla or both.

4. The method of claim 1, whereby the DKK polypeptide is Dkk1 polypeptide.

5. The method of claim 1, whereby the subject is susceptible to keloid formation.

6. The method of claim 1, whereby contacting comprises direct delivery of the DKK polypeptide to the matured hair follicle cell.

7.-11. (canceled)

12. The method of claim 7, whereby the DNA construct is a plasmid, a phagemid or an expression cassette.

13. A method of modulating hair growth in a post-natal subject without damaging skin or damaging a hair follicle, comprising the step of inhibiting Wnt/beta-catenin signaling in a matured hair follicle cell of the subject with a polypeptide capable of binding to LRP-family, Wnt-associated receptor, thereby modulating hair growth in a post-natal subject.

14. The method of claim 13, whereby the inhibiting Wnt/beta-catenin signaling comprises blocking a LRP-family, Wnt-associated receptor.

15. The method of claim 13, whereby the inhibiting Wnt/beta-catenin signaling comprises contacting the matured hair follicle cell with a DKK polypeptide or a DNA construct comprising the nucleic acid sequence encoding said DKK polypeptide.

16. The method of claim 13, whereby the hair follicle cell comprises a hair shaft, a dermal papilla or both.

17. The method of claim 15, whereby the DKK polypeptide is a Dkk1 polypeptide.

18. The method of claim 13, whereby the subject is susceptible to keloid formation.

19. (canceled)

20. The method of claim 15, whereby the DNA construct is a plasmid, phagemid, an expression cassette or their combination.

21. (canceled)

22. A method of temporarily inhibiting hair growth or temporarily removing hair on a subject without damaging skin or damaging a hair follicle, comprising the step of contacting skin of the subject with a topical composition comprising a DKK polypeptide or its peptidomimetic, thereby blocking LRP-family, Wnt-associated receptor in a mature hair follicle cell in the skin, thus temporarily removing hair on a subject.

23. (canceled)

24. The method of claim 22, whereby the composition inhibits Wnt/beta-catenin signaling.

25. The method of claim 22, whereby the composition comprises a DNA construct encoding a DKK polypeptide.

26. The method of claim 22, whereby the DNA construct is a plasmid, a phagemid or an expression cassette.

27. The method of claim 22, whereby the DKK polypeptide is Dkk1 polypeptide.

28. A method of inhibiting hair growth in a mature hair follicle, comprising the step of inducing the expression of a secreted Wnt inhibitor in a hair follicle cell, thereby inhibiting hair growth in a hair follicle.

29. (canceled)

30. The method of claim 28, whereby inducing the expression of a secreted Wnt inhibitor is transiently inducing the expression of a secreted Wnt inhibitor.

31. The method of claim 28, further comprises the step of transfecting the hair follicle cell with a vector comprising a DNA construct encoding the secreted Wnt inhibitor.

32. The method of claim 31, whereby the vector comprises an inducible promoter or a constitutively active promoter.

33. The method of claim 31, whereby the vector is a plasmid, a phagemid or an expression cassette.

34. The method of claim 28, whereby the Wnt inhibitor is expressed in an outer root sheath cell within the hair follicle.

35. The method of 28, whereby the Wnt inhibitor is expressed in a stem cell within the hair follicle.

36. The method of claim 28, whereby the secreted Wnt inhibitor is a Dkk polypeptide.

37. The method of claim 36, whereby the Dkk polypeptide is Dkk1 polypeptide.

38.-46. (canceled)

47. A method of reversibly inhibiting the proliferation of a hair follicle cell in a post-natal subject without increasing cell death of the hair follicle cells, comprising the step of contacting the hair follicle cell with a DKK polypeptide or its peptidomimetic analog, thereby reversibly inhibiting the proliferation of a hair follicle cell in a post-natal subject.

48. The method of claim 47, whereby inhibiting the proliferation of a hair follicle cell is during anagen of the hair cycle.

49. The method of claim 47, whereby inhibiting the proliferation of a hair follicle cell comprises reducing Cyclin D1 level, inhibiting BMP6 expression, or both.

50. The method of claim 47, whereby contacting the hair follicle cell comprises contacting a LRP-family, Wnt-associated receptor in the hair follicle cell.

51.-56. (canceled)

57. A method of reversibly inhibiting hair growth on a hair follicle in a post-natal subject without alteration in stem cell maintenance in the hair follicle, comprising the step of contacting the hair follicle during anagen phase of the hair follicle cell cycle with a DKK polypeptide or its peptidomimetic analog, thereby reversibly inhibiting hair growth on a hair follicle.

58. The method of claim 57, whereby inhibiting hair growth on the hair follicle comprises inhibiting the proliferation of a hair follicle cell

59. The method of claim 58, whereby inhibiting the proliferation of a hair follicle cell comprises reducing Cyclin D1 level, inhibiting BMP6 expression, or both.

60. The method of claim 57, whereby contacting the hair follicle cell comprises contacting a LRP-family, Wnt-associated receptor in the hair follicle cell.

61.-67. (canceled)

68. A method of maintaining a bald spot on a skin in a post-natal subject without damaging skin or damaging a hair follicle, comprising the steps of:
(a) contacting a LRP-family, Wnt-associated receptor in a matured hair follicle cell within the bald spot with a DKK polypeptide or its peptidomimetic analog; and
(b) removing hair within the spot, thereby maintaining a bald spot on a skin in a post-natal subject.

69. The method of claim 68, whereby maintaining a bald spot is done for a predefined period.

70. The method of claim 68, whereby maintaining a bald spot comprises continuous or periodical contacting of the LRP-family, Wnt-associated receptor in a matured hair follicle cell within the bald spot with a DKK polypeptide or its peptidomimetic analog.

71. The method of claim 68, whereby the hair follicle cell comprises a hair shaft, a dermal papilla or both.

72. The method of claim 68, whereby the DKK polypeptide is Dkk1 polypeptide.

73. The method of claim 68, whereby the subject is susceptible to keloid formation.

74. The method of claim 68, whereby contacting the matured hair follicle cell with a DKK polypeptide comprises directly delivering the DKK polypeptide to the mature hair follicle cell.

75. The method of claim 68, whereby the contacting comprises inducing the expression of a Dkk polypeptide.

76. The method of claim 75, whereby inducing the expression of a Dkk polypeptide comprises transiently inducing the expression of a Dkk polypeptide.

77. The method of claim 75, whereby contacting further comprises the step of transfecting the hair follicle cell with a vector comprising a DNA construct encoding Dkk polypeptide.

78. The method of claim 77, whereby the vector comprises an inducible promoter, a constitutively active promoter, or both.

79. The method of claim 77, whereby the vector is a plasmid, a phagemid or an expression cassette.

80. A method of reversibly inhibiting hair cortex formation in a post-natal subject without damaging skin or damaging a hair follicle, comprising the step of contacting a LRP-family, Wnt-associated receptor in a matured hair follicle cell with a DKK polypeptide or its peptidomimetic analog, thereby reversibly inhibiting hair cortex formation in a post-natal subject.

81.-97. (canceled)

98. A method of treating, inhibiting, suppressing or ameliorating symptoms associated with hirsutism or hypertrichosis in a subject, comprising the step of administering to the subject a composition comprising a DKK polypeptide, its peptidomimetic analog or a DNA construct encoding a secreted Wnt inhibitor, thereby binding a LRP-family, Wnt-associated receptor and reversibly inhibiting hair growth in the subject.

99.-103. (canceled)

104. The method of claim 98, whereby said inhibiting is transiently or reversibly inhibiting.

105. The method of claim 98, whereby said hair follicle cell comprises a hair shaft, a dermal papilla or both.

106. The method of claim 98, whereby said DKK polypeptide is Dkk1 polypeptide or its peptidomimetic.

107. The method of claim 98, whereby administering comprises direct delivery of said DKK polypeptide to a mature hair follicle cell.

108. The method of claim 98, whereby the DNA construct encoding the secreted Wnt inhibitor comprises a nucleic acid sequence encoding DKK polypeptide.

109. The method of claim 98, whereby said DNA construct is a plasmid, phagemid, an expression cassette or their combination.

110. The method of claim 98, whereby the DNA construct further comprises an inducible promoter.

111. (canceled)
